(19) United States
(12) Patent Application Publication
(10) Pub. No.: US 2022/0275318 A1
(43) Pub. Date: Sep. 1, 2022

(54) BIOREACTOR SYSTEMS, AND RELATED METHODS AND APPARATUS

(71) Applicant: Melonfrost, Inc., Brooklyn, NY (US)

(72) Inventors: Samuel R. Levin, Brooklyn, NY (US); Lucas J. Harrigan, Brooklyn, NY (US); Sean R. White, Brooklyn, NY (US); Loren K. Amdahl-Culleton, Brooklyn, NY (US)

(21) Appl. No.: 17/683,274

(22) Filed: Feb. 28, 2022

Related U.S. Application Data
(60) Provisional application No. 63/154,794, filed on Feb. 28, 2021.

(51) Int. Cl. C12M 1/00; C12M 1/06; C12M 1/02

(52) U.S. Cl. CPC C12M 23/14; C12M 27/02; C12M 41/18; B01F 2101/44

(57) ABSTRACT

A bioreactor system can include one or more bioreactor groups, where each group has a plurality of bioreactor clusters and a gantry shared by the plurality of bioreactor clusters. Each bioreactor cluster can include one or more manifolds and a plurality of bioreactor units. Each bioreactor unit can include a well, a lid covering the well, a waste valve to control a flow rate of waste out of the well, and one or more dispensing valves to control flow rates of dispensing one or more materials into the well. The gantry can include one or more sensors and a movable gantry head with one or more sensor probes configured to selectively sense properties of contents of respective bioreactor units included in the plurality of bioreactor units.

### CROSS-REFERENCE TO RELATED APPLICATIONS

This application claims the priority and benefit under 35 U.S.C. § 119(e) of U.S. Provisional Patent Application No. 63/154,794, titled “Bioreactor Systems, and Related Methods and Apparatus” and filed on Feb. 28, 2021, which is hereby incorporated by reference herein in its entirety. In addition, the subject matter of this disclosure is related to the subject matter of U.S. patent application Ser. No. 17/465,291 titled “Machine Learning and Control Systems and Methods for Learning and Steering Evolutionary Dynamics” filed on Sep. 2, 2021, which is hereby incorporated by reference herein in its entirety.

### TECHNICAL FIELD

The present disclosure generally relates to a bioreactor system suitable for using machine learning and optimal control techniques to learn and steer evolutional dynamics.

### BACKGROUND

Optimizing (e.g., improving) features of biological systems is a major goal in a number of industries including agriculture, energy, materials, and health care (e.g., medicine). Farmers try to maximize yields, biofuel developers try to maximize efficiency or minimize waste, materials companies try to optimize chemical ratios, and medical research scientists try to minimize pathogenicity and virulence.

Conventional approaches to optimization (e.g., improvement) of biological systems include genetic modification and editing, artificial selection, and environmental optimization. For example, biotechnology companies may identify useful genes and move them to a new species, or delete undesirable genes that are already present. Breeders may hand-pick the most desirable crops, and propagate them over and over again, thereby accentuating a trait. More recently, artificial intelligence (AI) companies have used machine learning to fine-tune the environment for an organism, e.g., by identifying an improved (e.g., optimal) nitrogen to phosphorous ratio for a tomato.

Artificial selection generally involves painstaking and brute force screening of individuals, selecting desirable ones, and repeating. Usually, artificial selection is performed to select individual traits that are expected to be useful, for example, a larger corn cob. Often, these individual traits scale nonlinearly at the population level, such that a field of corn plants with larger cobs actually produces less overall corn. Further, performing artificial selection without changing the environment can push the population away from a fitness optimum, such that, once the breeding program stops, the gains are reversed. Many of the early gains of genetically modified organisms (GMOs) were reversed in 20-30 years by evolution.

Environmental optimization, while a promising new tool, is limited in how much it can be expected to improve organisms. Focusing on which features of the environment are best for a given organism tends to lead to outcomes that are limited by the total potential of that organism's genotype. True biological optimization through modification of both the environment and the genome is potentially a much more powerful approach.

### SUMMARY

Fortunately, a potential alternative exists which can be controlled to solve these problems: evolution. In general, biological systems are both the products of evolution and constantly undergoing it. Evolution can push organisms in undesirable (from a human perspective) directions. For example, it can drive bacteria to become more virulent, or push crops to lower yields. Importantly, though, evolution does so in a much more robust way than current human techniques: it can select on the entire population, it can use random mutations to generate good genes that human engineers generally aren't able to efficiently identify, it tends to incorporate genes that work well in concert with the rest of the organism, it can continually check the qualities of every single individual in a population, at every instance, with perfect precision, and it can find solutions that are stable against perturbations. In principle, controlling evolutionary processes to choose desired qualities in a population can yield the most powerful biological optimizing tool to date.

The challenge is that evolution is generally slow, unpredictable, and doesn't optimize for human goals. It is generally difficult to predict what mutations will arise, what selection pressures new environments will impose, or which amongst many possible higher-fitness routes a population will pursue. The dynamics of evolutionary systems have long been considered far too complex to be precisely controlled by humans. However, the inventors have recognized and appreciated that suitable machine learning and evolutionary modeling techniques can be used to learn the relationship between environmental conditions and evolutionary pathways for various types of organisms. Some examples of such techniques are described in U.S. patent application Ser. No. 17/465,291 titled “Machine Learning and Control Systems and Methods for Learning and Steering Evolutionary Dynamics.” Given enough data, techniques for learning and steering evolutionary dynamics can be used to learn the relationship between environmental conditions and evolutionary pathways, and institute an optimal control approach to drive an organismal population in a controlled environment to an evolutionarily stable equilibrium that also improves (e.g., optimizes, maximizes, etc.) an attribute of (human) interest.

The inventors have recognized and appreciated that conventional bioreactors generally do not provide a suitably controlled environment and/or enough data for using suitable evolutionary learning and steering techniques to efficiently learn the evolutionary dynamics of an organismal population and control the population's environment such that the population is efficiently driven to an evolutionarily stable equilibrium that improves (e.g., optimizes) an attribute of interest. In general, one innovative aspect of the subject matter described in the present disclosure can be embodied in a bioreactor system (e.g., a massively parallel bioreactor system) suitable for learning and steering the evolutionary dynamics of a biological system.

According to an aspect of the present disclosure, a bioreactor system includes one or more bioreactor groups. Each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. The gantry includes one or more sensors and a movable gantry head with one or more sensor probes configured to selectively sense properties of contents of respective bioreactor units included in the plurality of bioreactor units.

According to another aspect of the present disclosure, a bioreactor system includes one or more bioreactor groups, where each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. Each bioreactor has an independent temperature control subsystem.

According to another aspect of the present disclosure, a bioreactor system includes one or more bioreactor groups, where each group has a plurality of bioreactor clusters and a gantry shared by the plurality of bioreactor clusters. Each bioreactor cluster includes a plurality of bioreactor units organized in rows. Each bioreactor unit has a well, a lid covering the well, a waste valve configured to control draining of waste out of the well, and one or more dispensing valves configured to control dispensing one or more materials into the well. Each bioreactor cluster further includes a top manifold shared by the plurality of bioreactor units included in the respective bioreactor cluster, and a plurality of bottom manifolds, each bottom manifold corresponding to a row of bioreactor units within the respective bioreactor cluster.

According to another aspect of the present disclosure, a bioreactor unit includes a well and a lid covering the well. The lid further includes a pierceable lid septum configured to permit injection of a seeding solution into the well. The bioreactor unit further includes one or more inlets configured to dispense materials into the well. Each inlet has a corresponding valve configured to control dispensing of material. The bioreactor unit further includes an outlet configured to drain waste from the well. The outlet has a corresponding valve configured to control draining of waste. The bioreactor unit further includes a fluid level sensor configured to sense a volume of contents of the well.

According to another aspect of the present disclosure, a bioreactor system includes a plurality of bioreactor units including a respective plurality of wells, a bioreactor controller board including a plurality of temperature sensors and optical density sensors corresponding, respectively, to the plurality of bioreactor units. Each temperature sensor is configured to sense a temperature of a well of the respective bioreactor unit and each optical density sensor is configured to sense an optical density of contents of the respective bioreactor unit. The bioreactor system further includes a plurality of lids configured to cover, respectively, the plurality of wells. Each lid includes a plurality of probe channels configured to extend downward from an upper surface of the lid into the respective well. Each of the probe channels is configured to guide an optical probe from the upper surface of the lid to a position proximate to a bottom of the probe channel.

According to another aspect of the present disclosure, a bioreactor system includes a plurality of bioreactor clusters, where each of the bioreactor clusters includes a plurality of bioreactor units and a gantry shared by the plurality of bioreactor clusters. The gantry includes a mobile gantry head, and a plurality of sensors. Each sensor includes a respective sensor probe, where the sensor probes are mounted on the mobile gantry head. The gantry is configured to selectively position the gantry head above each bioreactor unit in the plurality of bioreactor clusters, insert the sensor probes into corresponding probe channels in a lid of the respective bioreactor unit, and sense properties of contents of the respective bioreactor unit.

According to another aspect of the present disclosure, a method for forming a bioreactor system includes forming one or more bioreactor groups, where each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. The gantry includes one or more sensors and a movable gantry head with one or more sensor probes configured to selectively sense properties of contents of respective bioreactor units included in the plurality of bioreactor units.

According to another aspect of the present disclosure, a method for forming a bioreactor system includes forming one or more bioreactor groups, where each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. Each bioreactor further includes an independent temperature control subsystem.

According to another aspect of the present disclosure, a method for forming a bioreactor system includes forming one or more bioreactor groups, where each group has a plurality of bioreactor clusters and a gantry shared by the plurality of bioreactor clusters. Each bioreactor cluster includes a plurality of bioreactor units organized in rows. Each bioreactor unit has a well, a lid covering the well, a waste valve configured to control draining of waste out of the well, and one or more dispensing valves configured to control dispensing one or more materials into the well. Each bioreactor cluster further includes a top manifold shared by the plurality of bioreactor units included in the respective bioreactor cluster, and a plurality of bottom manifolds, each bottom manifold corresponding to a row of bioreactor units within the respective bioreactor cluster.

According to another aspect of the present disclosure, a method for forming a bioreactor unit includes forming a well, forming a lid covering the well, where the lid further includes a pierceable lid septum configured to permit injection of a seeding solution into the well; forming one or more inlets for dispensing materials into the well, where each inlet has a corresponding valve configured to control dispensing of material; forming an outlet for draining waste from the well, where the outlet has a corresponding valve configured to control draining of waste; and forming a fluid level sensor for sensing a volume of contents of the well.

According to another aspect of the present disclosure, a method for forming a bioreactor system includes forming a plurality of bioreactor units including a respective plurality of wells, forming a bioreactor controller board, where the bioreactor cluster board includes a plurality of temperature sensors and optical density sensors corresponding, respectively, to the plurality of bioreactor units. Each temperature sensor is configured to sense a temperature of a well of the respective bioreactor unit and each optical density sensor is configured to sense an optical density of contents of the respective bioreactor unit. The method further includes forming a plurality of lids, the plurality of lids being configured to cover, respectively, the plurality of wells, where each lid includes a plurality of probe channels configured to extend downward from an upper surface of the lid into the respective well, and each of the probe channels is configured to guide an optical probe from the upper surface of the lid to a position proximate to a bottom of the probe channel.

According to another aspect of the present disclosure, a method for forming a bioreactor system includes forming a plurality of bioreactor clusters, where each of the bioreactor clusters includes a plurality of bioreactor units, and a gantry shared by the plurality of bioreactor clusters. The gantry includes a mobile gantry head and a plurality of sensors, where each sensor includes a respective sensor probe. The sensor probes are mounted on the mobile gantry head. The gantry is configured to selectively position the gantry head above each bioreactor unit in the plurality of bioreactor clusters, insert the sensor probes into corresponding probe channels in a lid of the respective bioreactor unit, and sense properties of contents of the respective bioreactor unit.

According to another aspect of the present disclosure, a method for operating a bioreactor system is provided. The bioreactor system includes one or more bioreactor groups, where each group has a plurality of bioreactor clusters and a gantry shared by the plurality of bioreactor clusters. Each bioreactor cluster includes a plurality of bioreactor units. Each bioreactor unit includes a well, a lid covering the well and having one or more channel windows, and a first set of sensors disposed on one side of the bioreactor unit. The gantry includes a second set of sensors and a movable gantry head with one or more sensor probes configured to selectively sense properties of contents of respective bioreactor units included in the plurality of bioreactor units. The method includes performing a first measurement of contents inside a target bioreactor unit using one of the first set of sensors disposed adjacent to the target bioreactor unit, and/or performing a second measurement of the contents inside the target bioreactor unit using one of the second set of sensors included in the gantry.

According to another aspect of the present disclosure, a method for operating a bioreactor system is provided. The bioreactor system includes one or more bioreactor groups, where each group has a plurality of bioreactor clusters. Each bioreactor cluster includes a plurality of bioreactor units and a plurality of tanks or reservoirs for storing resources and waste materials. Each bioreactor unit includes a well, a lid covering the well, a waste valve configured to control draining of waste out of the well, and one or more dispensing valves configured to control dispensing of one or more materials into the well. The method includes dispensing one of the one or more materials into a well of a target bioreactor unit through controlling the waste valve and the one or more dispensing valves of each bioreactor unit, and/or draining waste from the well of the target bioreactor unit through controlling the waste valve and the one or more dispensing valves of each bioreactor unit.

The details of one or more embodiments of the subject matter described in this specification are set forth in the accompanying drawings and the description below. Other features, aspects, and advantages of the subject matter will become apparent from the description, the drawings, and the claims.

The foregoing Summary, including the description of some embodiments, motivations thereof, and/or advantages thereof, is intended to assist the reader in understanding the present disclosure, and does not in any way limit the scope of any of the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure. In the following description, various embodiments of the present disclosure are described with reference to the following drawings, in which:

FIG. 1 is an architecture-level diagram of a bioreactor system, according to some embodiments;

FIG. 2 depicts a bioreactor unit, according to some embodiments;

FIG. 3A depicts a bioreactor well, heat spreader, and TEC assembly, according to some embodiments;

FIG. 3B illustrates a simulation result of individual bioreactor units with temperature controlled through TEC heating and cooling, according to some embodiments;

FIG. 4 depicts a cross-sectional view of a bioreactor unit's stirring mechanism, according to some embodiments;

FIG. 5 depicts a lid for a bioreactor well, according to some embodiments;

FIG. 6A depicts a cross sectional view of a bioreactor unit, according to some embodiments;

FIG. 6B depicts a side view of a bioreactor unit, according to some embodiments;

FIG. 6C depicts fluid handling manifold mounted valves, according to some embodiments;

FIG. 6D depicts a software/firmware stack for a bioreactor board, according to some embodiments;

FIG. 7A depicts top and bottom views of a bioreactor cluster, according to some embodiments;

FIG. 7B depicts a schematic diagram of a bioreactor cluster, according to some embodiments;

FIG. 8 depicts a part of a bioreactor board, according to some embodiments;

FIG. 9A depicts a top manifold liquid pumping block diagram, according to some embodiments;

FIG. 9B depicts a bottom manifold liquid pumping block diagram, according to some embodiments;

FIGS. 10A, 10B, 10C, and 10D collectively depict a bioreactor cluster control board, according to some embodiments;

FIG. 11 depicts cluster controller board software modules, according to some embodiments;

FIG. 12 depicts a gantry system, according to some embodiments;

FIG. 13A depicts a layout of sensors in a sensor pod of a gantry, according to some embodiments;

FIG. 13B depicts a fiber optic sensor probe for a spectrometer, according to some embodiments;

FIG. 14 depicts oxygen management connections at a bioreactor station level, according to some embodiments;

FIG. 15 depicts a block diagram of high level bioreactor system architecture, according to some embodiments;

FIG. 16 depicts a high level software architecture of a bioreactor system, according to some embodiments;

FIG. 17 depicts a network between a master controller and bioreactor clusters, according to some embodiments; and

FIG. 18 depicts a diagram of an exemplary computer system that may be used in implementing some embodiments of the systems and methods described herein.

### DETAILED DESCRIPTION

The subject matter of the present disclosure will now be described more fully hereinafter with reference to the accompanying drawings, in which exemplary embodiments of the present disclosure are shown. The present disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

As embodied and broadly described herein, the present disclosure relates to a bioreactor system suitable for using machine learning and optimal control techniques to learn and steer evolutional dynamics. To achieve such functions, the disclosed bioreactor system is configured in a modular fashion that allows bioreactor units included in the system to be scaled to an appropriate size to accommodate the size and scale suitable for machine learning and optimal control techniques to learn and steer evolution dynamics. For example, the bioreactor system can be a large scale (e.g., “massively parallel”) bioreactor system that includes a large number (e.g., 1,000, 5,000, 10,000, etc.) of individual bioreactors capable of applying selective pressures to populations of organisms grown inside individual bioreactors, so that sufficient data can be collected to learn and steer the evolutionary dynamics of the organisms.

A skilled artisan will appreciate that the challenges associated with organizing and operating a large number of conventional bioreactor systems (e.g., hundreds or thousands of bioreactor units) in a controlled manner are potentially staggering, not to mention the cost associated with manufacturing this large number of conventional bioreactor systems and the complexity of controlling various growing conditions of this number of conventional bioreactor systems in a unified manner. The present disclosure is aimed at addressing these concerns by providing a large scale bioreactor architecture that organizes the bioreactors hierarchically into multi-well plates or clusters, which are further stacked into stations so that a large number of bioreactors can be organized to a reasonable size and scale, allowing the system to efficiently control each of the individual bioreactors. A large scale bioreactor system organized in accordance with such a bioreactor architecture can be referred to as a “massively parallel” bioreactor system.

In addition, certain other techniques and principles may be used in the design and construction of a large number of bioreactors in accordance with the bioreactor architecture described herein, to save cost and address other concerns that can arise when a large number of bioreactors are co-located and operated together. For example, clustering and resource sharing techniques can be used in a massively parallel bioreactor system to reduce per-well manufacturing costs and operating costs relative to conventional bioreactor systems. For example, bioreactor wells can be grouped into clusters, and the wells within each cluster can share certain components of the bioreactor system. In one example, all the bioreactor units in a cluster can be served by a shared mobile gantry on which certain sensors and/or dispensing components are mounted, such that those components can be shared among the bioreactor units in a cluster by moving the gantry across these bioreactor units, rather than replicating such components on a per-well basis, thereby greatly reducing the manufacturing and operating costs associated with these components.

According to some embodiments, each bioreactor unit includes a well (e.g., vessel) for holding an organismal population, a plurality of input and output channels (e.g., media channel(s), waste channel(s), sensor channels, communication channels, etc.) coupled (e.g., connected) to the well and bioreactor unit controlling components (e.g., electronics and/or software or firmware). The well can be formed in a manifold. The channel(s) can be formed in manifold(s), and can be in fluidic communication with the well. The sensor channels can be formed in the manifold(s). By forming these components in the manifold(s), the manufacturing and operation costs can be further reduced. In addition, by molding the media/waste channels into the manifold, instead of using disposable tubing as in other bioreactor systems, the efficiency of cleaning these media/waste channels used for fluid circulation and/or waste removal and the stability of these channels formed in the manifold(s) can be improved.

In many cases, it is highly beneficial for the bioreactor system's sensors to avoid disturbing or physically interacting with organisms growing inside bioreactor wells. Thus, in some embodiments, non-invasive mechanisms for taking measurements are used in the disclosed bioreactor system. For example, an optical density (OD) sensor in optical communication with a light pipe or light column partially submerged in a well's contents (e.g., medium) can take non-invasive measurements without disturbing the well's organismal population.

In addition, maintaining stable and/or acceptable environments within individual bioreactor units for this large number of bioreactor units can be challenging but vital to steering the evolution of organismal populations in a bioreactor system. For example, adequately controlling the temperatures of individual bioreactor units independently in a massively parallel bioreactor system can be difficult. The bioreactor system described in the present disclosure addresses this concern, in part, by including enough heating and cooling capacity in thermoelectric coolers (TECs) co-located with the individual bioreactor wells to counteract thermal loads from neighboring wells.

In some embodiments, machine learning-based techniques can be utilized to improve the operation of the disclosed bioreactor system. For example, for a massively parallel bioreactor system configured to maintain optimal (or near-optimal) selection pressures within the organismal populations in the individual bioreactors, expected changes in the optical density of the media within the bioreactor units can be forecast. The rates at which seed or other media are dispensed into the wells can be then controlled based on such forecasts, such that the desired population size and selection pressure are maintained within each organismal population.

In some embodiments, the disclosed bioreactor system can also use software-driven systems to schedule, monitor, control and/or distribute resources. In an example, the bioreactor system can autonomously or semi-autonomously manage certain processes and/or operations (e.g., periodic measurements and/or dispensing, etc.), which can allow more accurate control of these processes or operations, especially when combined with certain built-in monitoring systems for certain feedback- or feedforward-based controls.

In some embodiments, the bioreactor units and/or clusters within the disclosed bioreactor system can use self-organizing techniques. For example, bioreactor units can use self-organizing techniques to register and/or establish their locations inside each bioreactor cluster. In some embodiments, bioreactor clusters can also be configured to self-organize and coordinate to share the use of a gantry system.

It is to be noted that the above described features and advantages for the disclosed bioreactor system are not all-inclusive, and many additional features and advantages will be apparent to one of ordinary skill in the art in view of the figures and the following detailed descriptions.

### Overview of the Bioreactor System

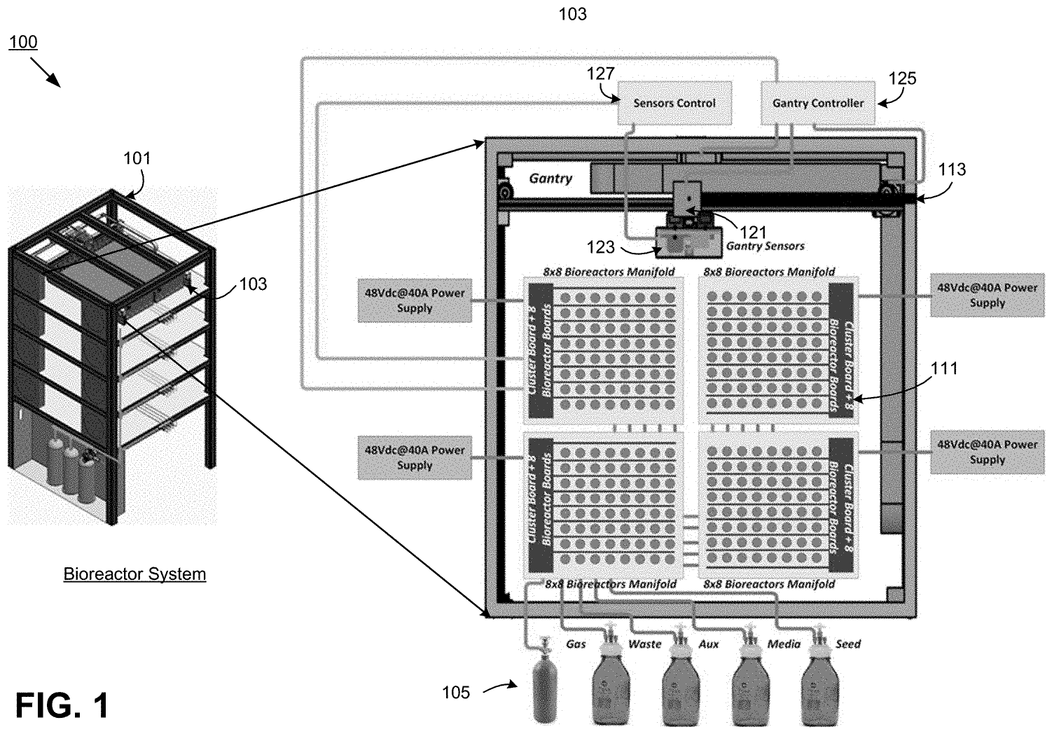

FIG. 1 depicts an exemplary bioreactor system 100, according to some embodiments. As illustrated in the left portion of the figure, the bioreactor system 100 includes a shelving unit 101 for holding multiple gantry systems 103, which are arranged in vertical layers in FIG. 1. Each gantry system 103 includes a number of bioreactor clusters 111 (e.g., 4 bioreactor clusters) and a shared gantry 113 (illustrated in the right portion of the figure). Each bioreactor cluster 111 includes a plurality of individual bioreactors organized in an array. For example, each bioreactor cluster can include an 8×8 bioreactor array that includes 64 individual bioreactors arranged in 8 rows and 8 columns. The foregoing examples are non-limiting. A gantry system 103 may include any suitable number of clusters (e.g., 1-1024 clusters), each of which may include any suitable number of bioreactors (e.g., 1-1024 bioreactors) arranged in any suitable configuration (e.g., square array, rectangular array, etc.).

As further illustrated in FIG. 1, each bioreactor cluster 111 further includes a cluster printed circuit board (e.g., “cluster circuit board,” “cluster PCB,” “cluster board,” or “cluster controller board”) and a certain number of bioreactor printed circuit boards (e.g., “bioreactor circuit boards,” “bioreactors PCBs,” “bioreactor boards,” or “bioreactor controller boards”) (e.g., 8 bioreactor boards). The cluster board can coordinate activity between the bioreactor units and the gantry 113, and provide an interface for high-level communication between the cluster and a master controller of the bioreactor system that manages all bioreactors units included in the system, as will be described in greater detail later. Each bioreactor board included in a bioreactor cluster can manage one or more bioreactor units (e.g., 8 bioreactor units in a row). To facilitate the communication with the cluster board and management of individual bioreactors, each bioreactor board can have a unique serial number that can be used to identify the board, and can store one or more unique IDs used to identify the bioreactor units managed by the board. The bioreactor board serial number and the bioreactor unit IDs may be stored, for example, in a computer-readable medium (e.g., digital memory) of the bioreactor board.

As illustrated in the right portion of FIG. 1, the gantry 113 includes a gantry head 121 and a certain number of sensor components 123 mounted on the gantry head 121. The gantry head 121 is movable according to a predefined pattern and can reach each bioreactor unit in the gantry system 103, so that the sensor components 123 mounted on the gantry head 121 can be shared among the bioreactor units. As illustrated in the figure, the gantry system 103 can also include a gantry controller 125 for controlling the motion of the gantry head 121 and a sensor controller 127 for controlling the operations of the sensor components 123. The specific details for gantry motion and sensor operations will be described in greater detail later.

As also illustrated in FIG. 1, the bioreactor system 100 can further include a certain number of tanks or reservoirs 105 for storing dispensable substances (e.g., nutrient sources) and/or storing waste (e.g., waste drained from the bioreactor units) during operation of the bioreactor system 100. For example, these tanks or reservoirs 105 can include a gas tank for providing gas (O₂, N₂, and/or others) to the bioreactors, a waste tank for collecting waste, an auxiliary tank for providing an auxiliary substance (“aux”) (e.g., bioreactor pH adjustment solution (e.g., “buffer solution”) to adjust the pH of bioreactor contents, a media tank for providing growth media to the bioreactors, and a seed reservoir for providing seeding organisms to the bioreactors.

Although not shown in FIG. 1, in some embodiments, the gantry head 121 can further include a seeding mechanism for seeding each bioreactor. Similar to the sensors mounted on the gantry head 121, by placing the seeding mechanism on the gantry, the cost of the bioreactor system can be further reduced.

The disclosed bioreactor system 100 may include additional components not illustrated in FIG. 1, such as bioreactor lids, fluid channels, light pipes, among others. In addition, while only one shelving unit or station is illustrated in FIG. 1, the disclosed bioreactor system is not limited to such configuration and can include two or more stations, depending on the experimental parameters and/or data collection specifications associated with the evolution dynamics analysis. The bioreactor system 100 will be described in greater detail hereinafter in an order beginning with individual bioreactors, then groups of individual bioreactors (e.g., row of bioreactors, bioreactor clusters), then bioreactor stations, and ending with a controlling system for the controlling operation of components included in the bioreactor system 100.

### 1. INDIVIDUAL BIOREACTORS (“WELLS”)

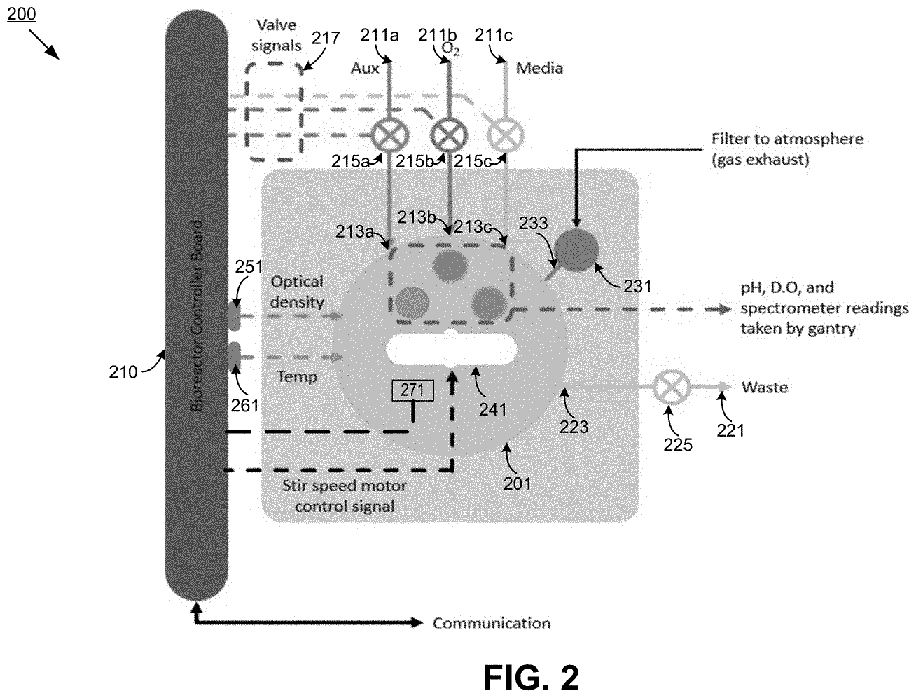

FIG. 2 illustrates an individual bioreactor 200 (or “bioreactor unit” 200) and associated accessories, according to some embodiments. As illustrated in the figure, an individual bioreactor 200 includes a well 201 that has a predefined shape or size. For example, suited for shear stress-sensitive or filamentous organisms, the well 201 can have a smooth sidewall, e.g., a round shape as illustrated in FIG. 1. The bottom of the well 201 can be flat-bottomed or can have a convex bottom surface in the center of the well 201 where a stirring bar is expected to reside. A convex bottom surface can reduce the friction force experienced by a rotating stirring bar 241, which also reduces the shear stress placed on organisms adjacent to the stirring bar.

The size of the well 201 can be also determined (e.g., selected) based upon the experimental parameters, e.g., the minimum number of organisms preferred to achieve sufficient data for evolution dynamics analysis. In real applications, the size of each individual bioreactor can be as large as a few milliliters to a few hundred milliliters, or as small as a few microliters to a few hundred microliters.

As further described in greater detail below, the sidewall and/or the bottom of the well 201 can further include ports for dispensing substances (e.g., fluids) (e.g., aux, growth media, and/or gas) and/or removing waste. The edges of these ports can be also smoothed to reduce the shear force experienced by the organisms growing inside the well 201.

In some embodiments, different materials can be used to manufacture wells 201 in the disclosed bioreactor system 100. These materials can include but are not limited to, certain types of transparent glasses or plastic materials that do not interfere with the organisms or growth media. For example, borosilicate glass or clear plastics such as polystyrene or polycarbonate can be used for manufacturing the wells 201.

In some embodiments, depending on the materials used to manufacture the wells, a bioreactor well can be further coated with certain materials on the bottom and/or sidewall surfaces to facilitate the growth of certain organisms. For example, the bottom of the well 201 can be further coated with certain biomaterials, such as collagen, extracellular matrix, laminin, fibronectin, certain mucopolysaccharides and the like for improved growth of certain organisms (e.g., virus growing inside cells that require cell attachment to the surfaces). More recently, certain synthetic nanofibers that mimic certain fibrillar topography can be also used to coat well bottom/surfaces to improve the growth of certain organisms. In some embodiments, with or without coating, a well bottom/surfaces can be also treated by using certain chemical or physical approaches to improve the growth of organisms inside a well. In one example, a high energy microwave plasma can be used to incorporate more oxygen onto a polystyrene surface of the well 201, rendering the surface more hydrophilic (wettable), suitable for growing certain organisms.

In some embodiments, each bioreactor unit 200 can be configured to ensure that the organisms in the bioreactor unit are not contaminated. For example, each bioreactor unit can be designed to be isolated. In an example, each bioreactor unit can be isolated from other bioreactor units and/or isolated from the external (e.g., ambient) environment.

In some embodiments, to maintain proper growth conditions and/or to facilitate selection pressures applied to growing organisms, each individual bioreactor 200 can be further equipped with certain controlling components for independently controlling growth conditions inside the bioreactor's well 201. The parameters that can be controlled include, but are not limited to, temperature (e.g., controlled by heating or cooling the bioreactor unit), organism concentration or optical density within the well (e.g., controlled by media addition), pH within the well (e.g., controlled by aux addition), mixing rate (e.g., controlled by actuating the stirring bar and setting the stirring speed), dissolved oxygen level within the well (e.g., controlled by adding O₂), etc., which individually or in combination can be used as the selection pressures for evolution dynamics learning and steering.

The controlling components for controlling the listed parameters include components for controlling contents of a well (e.g., components for dispensing materials into a well, components for removing waste from a well, components for controlling growth volume of a well, etc.), components for controlling growth conditions/processes within a well (e.g., temperature control apparatus, stirring mechanism, headspace gassing mechanism, etc.), and components for sensing different parameters to facilitate the control of these parameters (e.g., sensors for probing temperature, pH, dissolved oxygen, optical density, fluid level, etc.). In some embodiments, these controls and/or sensor components interact with each other. For example, the stirring rate can affect the dissolved oxygen level. In some embodiments, even when there are interactions among these controlling parameters, the control loops in the software and/or firmware in the disclosed bioreactor system 100 can be configured to maintain the specified target levels for each individual parameter. The specific functions of each component will be described in greater detail below in sections 1.1-1.3.

### 1.1 Components to Control Contents of a Well

Still referring to FIG. 2, the components to control contents of a well include, but are not limited to, components to dispense materials into a well, components to remove waste from a well, and components for controlling liquid volume or depth of liquid inside the well during organism growth, as further described in greater detail below.

1.1.1 Components to Dispense Materials into a Well

As described earlier, to facilitate dispensing materials, fluidic channels can be formed in a manifold to facilitate cost-effective dispensing of materials (e.g., fluid) to each individual bioreactor unit and to remove waste from each individual bioreactor unit without using a large number of tubes and fluidic fittings as other existing bioreactors do. Forming the fluidic channels in the manifold can greatly reduce the risk of leaks, reduce assembly time, and also increase the density of the bioreactor units so that more units can fit in a set space. In addition, this configuration can also allow users to more efficiently clean the fluid channels used for fluid circulation and/or waste removal. Further, channels formed in a manifold can be significantly more durable than disposable tubing used by other bioreactor systems. As described in further detail below, the substance in each fluidic channel may be dispensed from a source reservoir or tank. In some embodiments, a pump (e.g., peristaltic pump) in combination with a liquid flow sensor may be used to precisely control the flow of fluid from the source. For example, flow sensors and pumps located at the entry to a cluster's network of fluidic channels can control and detect the flow rate of each fluid into the cluster's fluidic channels.

In some embodiments, each bioreactor unit can include valves 215 (e.g., binary valves) to control the delivery (e.g., flow rate) of fluids (e.g., media, aux, or O₂) from the fluidic channels 211 into the bioreactor well 201. In one example, the fluid channels for a bioreactor unit 200 can include fluid channels to dispense aux, oxygen (02), and media, as illustrated by flow channels 211a, 211b, and 211c in FIG. 2. Each channel 211a/211b/211c can have an opening or port 213a, 213b, or 213c (which can be also referred to as inlet) at the sidewall of a well 201. As also illustrated in FIG. 2, each fluid channel 211a/211b/211c can be further equipped with a valve (e.g., 215a, 215b, or 215c) for control of dispensing the corresponding material into the well 201. For example, the valves 215a/215b/215c can receive valve signals 217 (e.g., from a bioreactor board 210) to determine when to open (e.g., partially or fully open) or close (e.g., partially or fully close) the valve so that a desired amount of fluid can be delivered to a well 201. In one example, to control the pH of a well 201, a precision binary valve 215a can be used to dispense quantities, including microliter quantities, of 1M HCl or NaOH or any other suitable buffer solution to the well 201. The valve controller (e.g., bioreactor board) may account for a suitable degree of hysteresis between the delivery of the buffer solution to the well and the measurement of the pH in the well to prevent overcompensating through excessive aux addition, although the skilled artisan can have some flexibility in controlling the volumes of aux dispensed to adequately control the well's pH.

In some embodiments, instead of maintaining pH at a desired setpoint (e.g., pH=7) for optimal organism growth, the pH of the growth media can be controlled to be other different values, so that a specified selection pressure can be applied to a growing population. Similarly, the valve controllers can control the dispensing of other materials (e.g., media, O₂) into the well 201 such that the amounts of corresponding substances in the well (e.g., concentration of nutrients or dissolved oxygen) are set or maintained at specified levels. Under certain circumstances, the dispensing of these materials can deviate from the dispensing rates associated with optimal organism growth, so that certain selection pressures can be applied. For example, different bioreactor units can be controlled to have different concentrations of one or more nutrients and/or different concentrations of dissolved oxygen, so that certain selection pressures can be applied to each bioreactor unit 200.

In some embodiments, to dispense a material (e.g., media, aux, or O₂) into a well 201 of a bioreactor unit 200, the valve 215 for the fluidic channel 211 for the target material for the well of interest is opened, and other well valves in the bioreactor unit's cluster are closed. For example, all other valves (215, 225) of the well of interest and all valves (215, 225) of all other wells in the cluster may be closed, such that while any one material is being dispensed into any one well in the cluster, that material may not be dispensed into any other well in the cluster via the channels 211, no other material may be dispensed into any well in the cluster via the channels 211, and waste may not be drained from any well in the cluster via the channels 221. Alternatively, all other valves 215 for fluid-dispensing channels 211 in the cluster may be closed, such that while any one material is being dispensed into any one well in the cluster, that material may not be dispensed into any other well in the cluster via the channels 211, and no other material may be dispensed into any well in the cluster via the channels 221, but waste may be drained from one or more wells in the cluster via the channels 221. Alternatively, all other valves 215 for the fluid-dispensing channels 211 for the target material in the cluster may be closed, such that while any one material is being dispensed into any one well in the cluster, that material may not be dispensed into any other well in the cluster via the channels 211, but one or more other materials may be dispensed into another well in the cluster via a channel 211, and/or waste may be drained from one or more wells in the cluster via the channels 221. In some embodiments, in each row of bioreactor units 201 in the cluster, no more than one bioreactor 201 may have the valve 215 for the fluid-dispensing channel 211 for the target material open at a given time, such that the target material can be dispensed to one bioreactor well per row via the channel 211 simultaneously. After opening the valve 215 for the fluidic channel 211 for the target material for the well of interest and closing the other valves, the corresponding pump for the fluid-dispensing channel 211 for the target material for the cluster is turned on. The flow sensor may measure the flow rate and thus determine how long the pump remains on to dispense the specified amount of material into the well.

While FIG. 2 illustrates three different fluid channels for aux, O₂, and media, the disclosed bioreactor system 100 is not limited to such configuration. In some embodiments, more or fewer fluid channels can be provided for each bioreactor unit 200. For example, under certain circumstances, there may be two fluid channels (e.g., aux1 and aux2) used for dispensing substances that control pH, with one channel specifically configured for dispensing acid and the other channel configured for dispensing base, so that the pH value of the growth media can be timely increased or decreased. In many situations, however, the growth of an organism tends to cause pH to drift only in one direction, e.g., cause pH to only increase or decrease. Accordingly, one aux channel can be enough for a run of the disclosed bioreactor system 100, since there may be only one type of organism growing in each run, and thus one type of aux (either base or acid) is sufficient. Additionally or alternatively, a buffer solution can be used to replace acid or base so that a single aux channel is sufficient to maintain the pH of the growth media within a proper pH range.

Under certain circumstances, if organism growth does not cause a pH change or if the growth of an organism is not affected by the pH change, the illustrated aux channel 211a can be excluded from the disclosed bioreactor unit 200, which further saves the space and simplifies the configuration of the whole bioreactor system 100.

1.1.2 Components to Remove Waste from a Well

In some embodiments, the bioreactor unit 200 may include a fluid channel 221 for removing waste from the well 201. Like the material-dispensing channels 211, the waste removal channel 221 can be formed (e.g., molded) into a manifold, rather than using disposable tubing. This waste removal channel 221 can also have an opening or port 223 at an outlet of a well 201. In some embodiments, the opening or port 223 can be disposed on the bottom of the well to facilitate waste removal. As illustrated, the waste removal channel 221 can be equipped with a valve (e.g., binary valve) 225.

In some embodiments, to remove the waste from a well 201, the cluster board for the well's cluster opens the waste removal valve 225 for the well of interest and closes other well valves in the cluster. For example, all other valves 215 of the well of interest and all valves (215, 225) of all other wells in the cluster may be closed, such that while waste is being drained from any one well in the cluster, material may not be dispensed into any well in the cluster via the channels 211 and waste may not be drained from any other well in the cluster via the channels 221. Alternatively, all valves 215 for fluid-dispensing channels 211 in the cluster may be closed, such that while waste is being drained from any one well in the cluster, material may not be dispensed into any well in the cluster via the channels 211, but waste may be drained from one or more other wells in the cluster via the channels 221. Alternatively, all other waste-removal valves 225 in the cluster may be closed, such that while waste is being drained from any one well in the cluster, material may be dispensed into one or more wells in the cluster via the channels 211, but waste may not be drained from any other wells in the cluster via the channels 221. After opening the valve 225 for the waste removal channel 221 for the well of interest and closing the other valves, the cluster board turns on the waste removal pump for the cluster. A flow sensor located at the outlet of the cluster's waste channel may check the flow rate and thus determine how long the pump remains on to remove the specified amount of waste from the well.

Some embodiments have been described in which, during operation, the bioreactor 100 automatically closes certain valves (215, 225) in a cluster when one or more other valves (215, 225) in the cluster are open. In some embodiments, during cleaning, the bioreactor 100 can open any suitable combination of valves (215, 225) in a cluster (e.g., all valves in the cluster) to facilitate a thorough cleansing of the channels (211, 221) and the bioreactor wells 201.

Although not illustrated in FIG. 2, in some embodiments, the valve 225 receives valve control signals from the bioreactor board. The valve control signals can control the valve 225 to open (to remove the waste from the well 201) and to close (e.g., when a specified amount of waste has been removed from the well 201).

In some embodiments, not just waste, but excess gas can be removed from the well 201 of a bioreactor unit 200. Accordingly, an exhaust valve (e.g., valve 231) can be included in a bioreactor unit 200. To improve stability, a fluid exhaust channel 233 can be formed (e.g., molded) into a manifold for each bioreactor unit 200. In some embodiments, to prevent the discharge of liquid through the exhaust channel together with the gas, the opening of the exhaust channel 233 can be adjacent to the top of the well 201. By placing the exhaust opening on the sidewall but not the lid of a well 201, it can be ensured that the motion of a gantry over a well is not affected when excess gas is released. In some embodiments, to prevent environmental or cross-contamination, a filter can be further installed within or at an end of exhaust channel 233. In some embodiments, the bioreactor unit 200 opens the exhaust valve to allow excess gas to leave the well 201 when the pressure within the well exceeds a specified threshold value. The threshold value for pressure with a bioreactor's well may be an evolutionary selection pressure.

1.1.3 Components to Control Volume of Contents inside a Well

In some embodiments, dispensing materials into a well and/or removing waste from a well can cause a change in the volume of the contents (e.g., growth media) inside a well 201. Accordingly, to facilitate maintenance of a suitable volume of contents (e.g., growth media) inside a well 201, a liquid level sensor 271 may be included in a bioreactor unit 200. Any suitable liquid level sensor may be used (e.g., a capacitive liquid level sensor). Such liquid level sensor can be configured to measure the media depth inside a well, so that the volume of the well's contents (e.g., growth media) can be determined. When the volume is determined to be lower than a specified threshold volume (e.g., if an excessive amount of waste is removed from a well 201), media can be added to the well. When the volume is determined to be higher than a specified threshold volume (e.g., when aux or media is added to a well to adjust growth conditions (e.g., adjust nutrient and/or organism concentration)), waste removal can be activated to remove a certain amount of waste from the well. It is to be noted that “waste” discussed here and throughout the specification can include any excess amount of growth media/organisms (e.g., any amount of growth media/organisms outside a target range for section pressure purposes) during the growth of organisms inside a well 201. For example, organisms inside a well can be deliberately removed as “waste” to hold density constant (or reduce density) if desired. Additionally or alternatively, when a run ends, “waste” can include the growth media and the organisms that remain in each well 201.

In some embodiments, the volume of growth media inside a well can be controlled without consulting the output from a liquid level sensor. For example, to stably maintain the volume of growth media inside a well, every time a certain amount of material is dispensed into a well 201, a corresponding amount of waste can be removed from the respective well. Similarly, if a certain amount of waste is removed (e.g., to control organism population inside a well), the same amount of growth media can be added into the same well right before (e.g., immediately before), during, or after (e.g., immediately after) the waste removal.

Under certain circumstances, even if there is no waste removal via a waste channel 221, a certain amount of media can be dispensed into each well 201 to counteract the evaporation of growth media (which can happen through gas release via exhaust channel 233 or through other means).

In some embodiments, besides volume control, certain other environmental conditions (e.g., temperature) or processes within the wells of the bioreactor unit can be controlled, as further described in detail in the next section.

1.2 Components to Control Conditions/Processes within a Well

### 1.2.1 Temperature Control Subsystems

Temperature control of a bioreactor unit 200 can be an important factor in maintaining a controlled environment for the unit's organismal population. In some embodiments, a bioreactor unit 200 can be designed without a fan. For example, the bioreactor unit 200 can include a passive cooling configuration or passive heat exchanger such as a cold finger and/or heat sink. In some embodiments, the bioreactor system can use an active cooling system instead, e.g., a fan can be used and/or a coolant can be circulated.

In some embodiments, certain manifold designs can be used to control the temperature of a bioreactor unit. For example, the use of suitable manifolds can provide a sufficient degree of thermal isolation and/or temperature control by decreasing the amount of physical material between wells while maintaining structural and fluidic thermal isolation and/or temperature control features (e.g., air gaps). In some embodiments, an insulating material can be added (e.g., to the sidewalls and/or bottom surfaces of the bioreactor wells 201, and/or to the manifold(s)) to improve the thermal isolation and/or temperature control of individual bioreactor wells while decreasing the impact of each well's temperature on adjacent wells. In some embodiments, to achieve a suitable level of thermal isolation and/or temperature control of bioreactor wells, the bioreactor pitch (distance between adjacent bioreactor wells) can be increased to increase the air gap between wells, the amount of insulating material between wells, and/or the amount of manifold substrate material (which can have a relatively high thermal resistance) between wells.

In some embodiments, the bioreactor wells 201 (like the channels 211) can be formed in the manifold. In some embodiments, the manifolds are diffusion bonded. In some embodiments, liquid cooling of the manifold can be used for controlling the temperature of the bioreactor well.

In some embodiments, a bioreactor unit 200 may include a thermoelectric cooler (TEC) suitable for controlling the temperature control of the well 201 by heating and/or cooling the well 201. A TEC operates according to the Peltier effect. The effect creates a temperature difference by transferring heat between two electrical junctions. A voltage is applied across joined conductors to create an electric current. When the current flows through the junctions of the two conductors, heat is removed at one junction and cooling occurs. Heat is deposited at the other junction. The most common application of the Peltier effect is cooling. However, the Peltier effect can also be used for heating or cooling (more generally, for controlling temperature). A DC voltage can be used to allow a TEC to control the temperature of a bioreactor well 201 within a target range.

In some embodiments, a “cold finger” can be further used to distribute the heating and cooling generated by a TEC to the region of the manifold (e.g., a “cup”) where the bioreactor well is located. In some embodiments, a heat sink and airflow at the backside of the TEC can be used to dissipate heat. In some embodiments, the bioreactor system can be configured to achieve thermal isolation and/or temperature control of bioreactor wells by including enough heating and cooling capacity in the TECs to counter thermal loads from neighboring wells.

In some embodiments, eddy currents can be induced from the spinning magnetic bars, creating a magnetic field in the cold finger. Accordingly, an evaluation can be performed during a design process to ensure that the overall operation of the bioreactor system is not negatively affected by the induced eddy currents when TECs and cold fingers (e.g. copper cold fingers) are included.

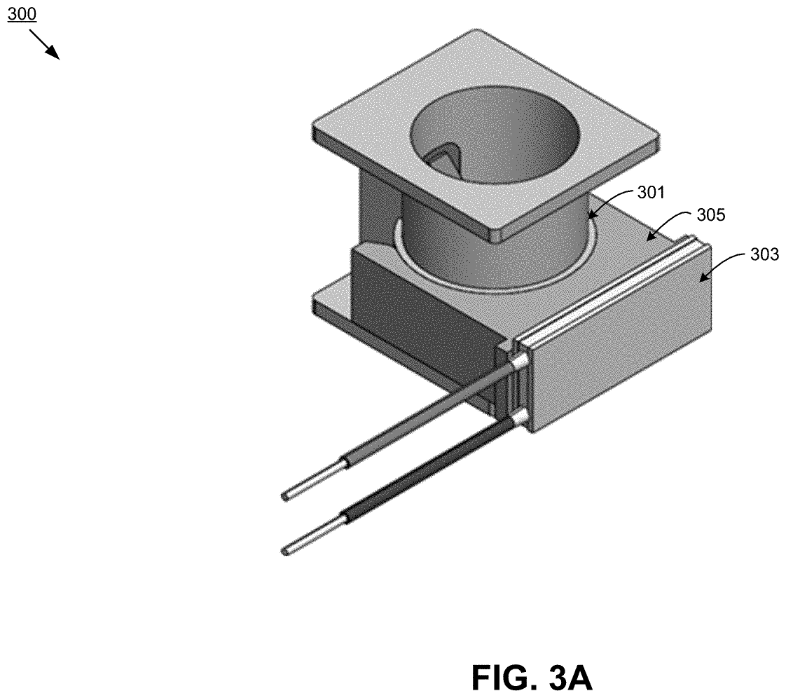

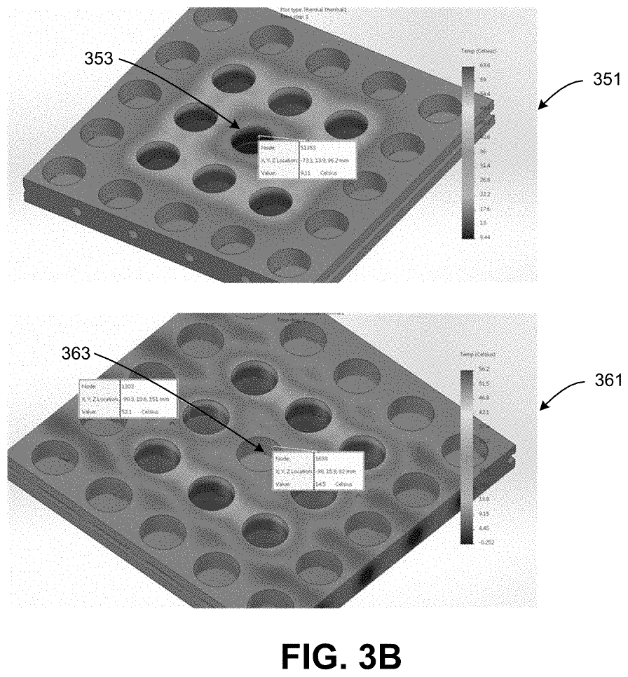

FIG. 3A illustrates a bioreactor unit 300 with a bioreactor well 301 (e.g., an injection molded bioreactor well 301), TEC assembly 303, and heat spreader 305 (e.g., copper heat spreader), according to some embodiments. The TEC assembly 303 can be located, for example, adjacent to one side of the bioreactor well 301, and heating/cooling generated by the TEC assembly 301 can be spread around the bioreactor well 301 through the heat spreader 305 or a cold finger or heat sink as described above.

In some embodiments, the process used for controlling TEC operation can be quite complex because of the nonlinearity introduced when the TEC current is reversed. In addition, heat can bleed back into the bioreactor units through the heat sinks when power is reduced or reversed. In some embodiments, the heating and cooling of the bioreactor system can be controlled using one or more neural networks. In an example, a neural network can be used to control the heating and cooling of a bioreactor unit achieved through TECs.

Suitable or optimal values for parameters of the TEC design (e.g., volume and/or spacing) can be determined using any suitable technique. For example, a thermal analysis can be performed to determine the TEC volume and/or spacing for the disclosed bioreactor system 100, so that the temperature of each bioreactor unit can be independently controlled. FIG. 3B illustrates a simulation result 351 of individual bioreactor units with temperature controlled through TEC heating and cooling, according to some embodiments. In particular, the simulation graph 351 shows a bioreactor unit 353 with a relatively low temperature, surrounded by bioreactor units with relatively high temperatures. As illustrated in the simulation graph 351, the temperature of a bioreactor unit 353 can be independently controlled through TEC heating and cooling. For comparison, a bottom simulation graph 361 shows temperature distribution for bioreactor units in which temperature control techniques other than TECs are used. In particular, the simulation graph 361 shows a scenario in which the temperature of a bioreactor unit 363 is not sufficiently isolated from the temperatures of the surrounding bioreactor units. As can be seen, the temperature of the bioreactor unit 363 is not well controlled (in particular, the temperature of bioreactor unit 363 is much higher than the target range), and the temperatures of the surrounding bioreactor units are somewhat lower than desired.

### 1.2.2 Stirring Mechanism

Mixing is another important factor that can affect organism growth. For example, when the bioreactor units are changing temperature, as when a controller initiates a temperature change to apply selection pressure, mixing can ensure a relatively uniform temperature throughout the contexts of the bioreactor well, and can also facilitate an accurate measurement of the temperature by the bioreactor unit's temperature sensor.

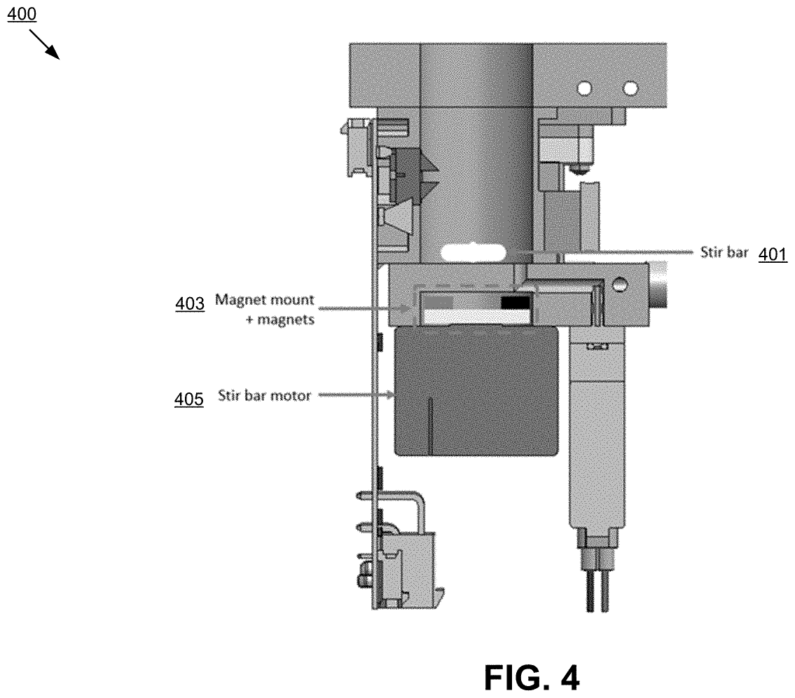

In some embodiments, mixing can be executed by a magnetic stir bar and stirring actuator. As illustrated by stir bar 241 in FIG. 2 and further by stir bar 401 in FIG. 4, a stir bar can be placed at the bottom of a bioreactor well 201. The stir bar, when activated by a stirring actuator (e.g., magnetic stirrer), can allow the contents of the well (e.g., growth media or O₂) to be mixed well during organism growth. The actuator can be used to set the speed of the stir bar 241 at a specified value within a predefined range, e.g., 150-1400 revolutions per minute (RPM). The exact speed that a stir bar is managed to rotate can be determined based on the volume of growth media, the content of the media, the size of the stir bar, and potentially the type of organisms that grow inside a well.

Referring to FIG. 4, a cross-sectional view of a bioreactor unit's stirring mechanism 400 is presented, according to some embodiments. In the example of FIG. 4, the stirring mechanism includes a magnetic stir bar 401, a magnetic mount and/or magnets 403, and a motor 405. Mixing is achieved with the magnetic stir bar 401 driven by a magnetic mount and/or magnets 403 positioned under the stir bar 401 (e.g., adjacent to the bottom of the well) and attached to the shaft of a stir bar motor 405. Besides general mixing, the stir bar motor 405 can also be used to increase or decrease the stirring rate of the stir bar 401, depending on the selective pressure chosen for mixing. More generally, the stirring mechanism 400 may include a stir bar, shafted impeller, or any other suitable mechanism suitable for stirring the contents of a bioreactor well.

### 1.2.3 Headspace Gassing for Aeration

In some embodiments, it can be useful to manage the release of gas from an organismal population. In one example, controlling the release of gas from individual bioreactor units can allow sufficient aeration for growing organisms. In addition, the release of gas from individual bioreactors can also one or more sensing devices to be shared among the bioreactor units in a cluster (e.g., in contrast to providing individual sensors for each bioreactor unit). As described in section 1.1.2, an exhaust valve 231 can be used to allow the outgassing of a well for sufficient aeration.

### 1.3 Components to Facilitate Sensing

To determine how a particular organismal population in a bioreactor unit is evolving (e.g., the direction of evolution in a phenotypic space, the direction of evolution according to a relevant evolutionary metric, etc.), the bioreactor system can include sensors to monitor the progress of organisms/organismal populations in the bioreactor system. In one example, the sensor output can be used in two ways. The first can be to maintain the current environment inside the bioreactor units. The second can be to generate a reward for the phenotypic change in an organismal population due to the evolution of a generation of organisms during an epoch.

Without limitation, one or more of the following sensors can be used:

1. Multi-wavelength fluorescent spectrometer

2. pH sensor

3. Dissolved oxygen (DO) sensor

4. Optical density (OD) sensor

5. Temperature sensor

6. Infrared sensor

7. UV sensor

Data can be collected by the sensors from time to time (e.g., on a periodic basis) and stored in a database. Additionally, data from a current run and previous runs on the bioreactor system can be collected. The data gathered can be used to determine how to control the selective pressures on subsequent generations of organisms. In an embodiment, although several example sensors are presented above, the bioreactor unit can be configured to use other sensors in addition to the list shown.

### 1.3.1 Lid for Optical Sensing

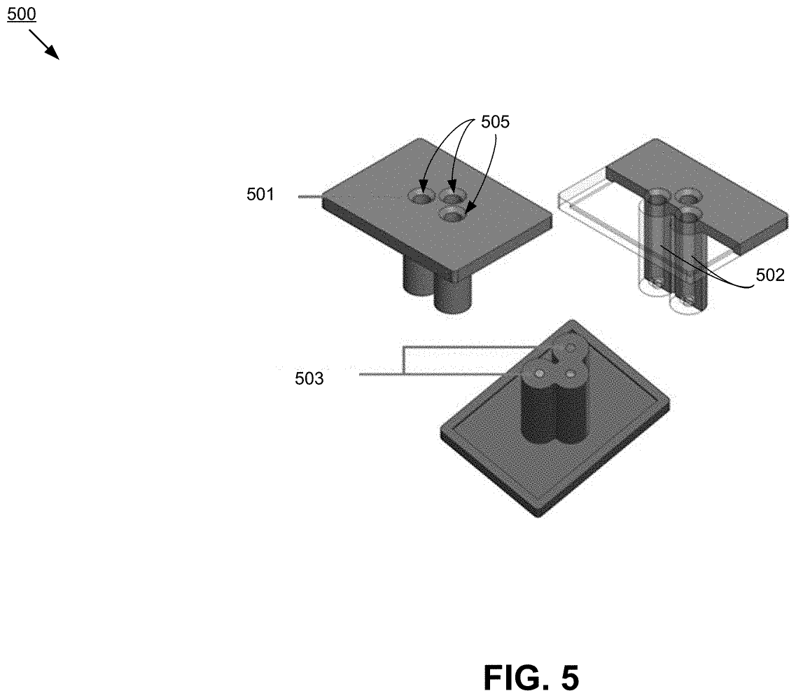

In the disclosed bioreactor system 100, the lids of the bioreactors wells can facilitate collection of sensor measurements by the sensor components controlled by the gantry (e.g., through the sensing probes disposed on the movable gantry head). For example, the spectrometer, pH, and dissolved oxygen measurements can be collected by the gantry sensors via optical interfaces in the lid assembly. Referring to FIG. 5, each bioreactor lid 501 can have three probe channels 505, each housing a transparent light window (e.g., small light pipe) 502 that allows the three above-mentioned measurements to be taken. The probe channels 505 configured to receive the sensor probes that collect the pH and dissolved oxygen readings can have respective sensing stickers 503 applied to the probe channel on the side of the light window that contacts the fluid contained in the bioreactor. The probe channels 505 can guide the sensing probes from the gantry head to their respective light windows.

The three probe channels 505 illustrated in FIG. 5 are meant for the insertion of the spectrometer, pH, and dissolved oxygen sensing probes from the gantry head when the gantry head is positioned above a bioreactor unit. Each probe channel 505 has an opening at the upper surface of the lid 501 through which the gantry head can insert a sensor probe into the probe channel. The other end of the probe channel is sealed and transparent and can be submerged in the well's media during normal organism growth. In some embodiments, to provide proper alignment between the optical sources and sensor probes, the light pipes for measuring spectrometer, pH, and dissolved oxygen can have a 45-degree internal reflection at the locations submerged in the sample. This configuration can allow the sensors to remain above the sample while the emitters and photosensors have a view of the sample through the light pipes.

In some embodiments, a color-changing sticker or liquid dot is placed on the transparent end or “window” of the probe channel configured to receive the gantry head's pH sensing probe to facilitate pH sensing. Likewise, in some embodiments, a color-changing sticker or liquid dot is placed on the transparent end or “window” of the probe channel configured to receive the gantry head's dissolved oxygen (DO) sensing probe to facilitate sensing of the DO level. The third probe channel can be configured to receive the gantry head's spectrometer probe, which can sense fluorescence.

In some embodiments, the lid assembly can be removably fastened to the bioreactor manifold. When necessary, the lid assembly can be removed from the corresponding bioreactor, for example, for cleaning and/or to replace probe dots or stickers. The specific functions of these sensing probes including their structures and functions will be described in greater detail in later sections related to the gantry system.

### 1.3.2 Sensors for Non-Invasive or Minimally-Invasive Sensing

The non-invasive and minimally-invasive sensing techniques applied to the disclosed bioreactor system 100 can include detection of optical density and temperature for a well 201 using non-invasive sensing techniques and detection of the liquid level inside the well using minimally-invasive sensing technologies. For the non-invasive detection of the optical density and the temperature, there is no direct contact between the growth media inside a well and the temperature sensor (or the optical density sensor) when measuring the temperature (or optical density) inside a well. For the minimally-invasive detection of the liquid level inside a well, a sensor probe can be placed inside the well 201, as further described in greater detail below.

### Optical Density Sensing

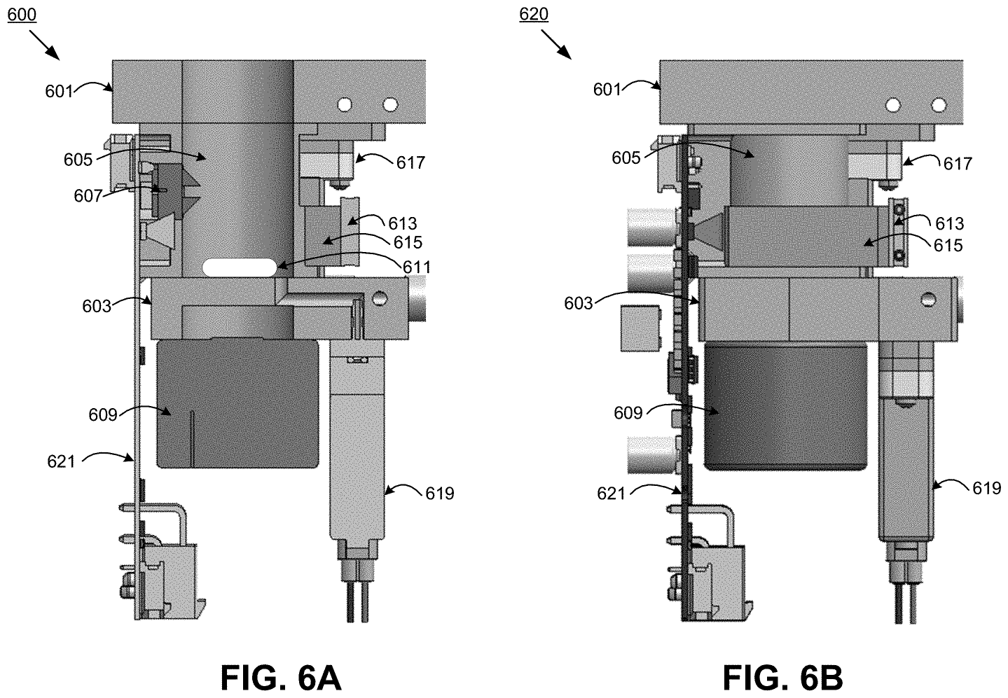

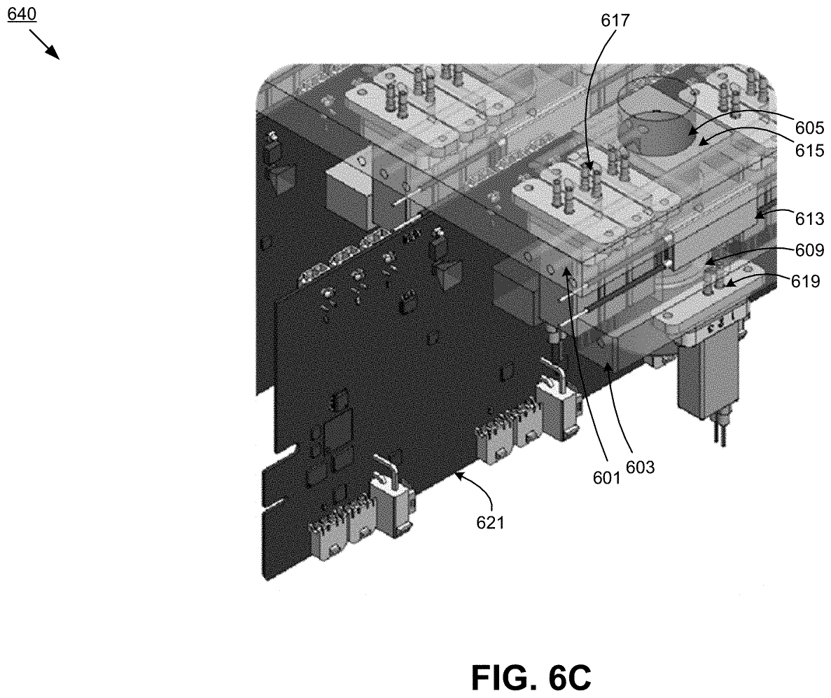

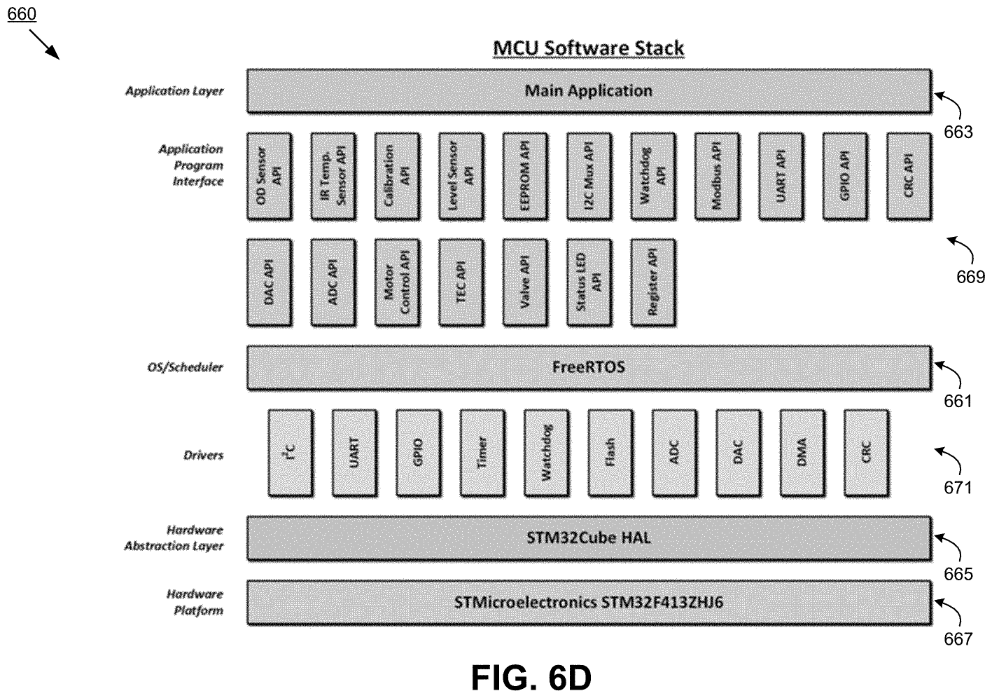

Referring to FIG. 2, each bioreactor unit 200 can include an optical density (OD) sensor 251. The OD sensor 251 can be controlled by the bioreactor unit's bioreactor board 210. Referring to FIG. 6A, the OD sensor 251 can include an optical density light pipe 607, which can be disposed on one side of the bioreactor well 605. In some embodiments, one end of the OD light pipe 607 penetrates through the side of the bioreactor well 605, such that the end of the OD light pipe 607 can be submerged in the contents of the well. The OD sensor 251 can be used to measure the optical density of organisms growing inside the well 605.

Measuring the OD is a common method to quantify the concentration of substances (Beer-Lambert law), since the light absorbance is proportional to the concentration of the absorbing particles in the sample. Optical density measurements can be used to determine the concentration of an organism (e.g., bacteria) in the growth media in a well 201. Under certain circumstances, if the OD reading indicates that the concentration of the organisms is too high (e.g., greater than a threshold concentration), a waste removal process can be activated to remove a portion of the growing organisms and, optionally, additional growth media can be dispensed into the well 201.

An OD sensor can quantify the optical density of a liquid sample by comparing the intensity of light that has passed through the liquid sample to the intensity of the light before it enters the sample. The sensing of OD, also known as “turbidity,” is generally performed using optical signals (light) at NIR (near-infrared) wavelengths insensitive to changes in media color. All particles that scatter NIR light are detected, including living and dead micro-organisms as well as micro-organisms debris. The OD measurement is particularly useful when the organisms are present in low densities (e.g., shortly after seeding), or when the organisms are of very small size (e.g., less than 1 μm) not easy to detect for capacitance-based instrumentation.

In some embodiments, the measurements provided by an OD sensor can be most accurate when a portion of the OD sensor (e.g., an end of the OD sensor light pipe 607) is fully submerged in the contents (e.g., media) of a bioreactor unit. In one example, the OD sensor light pipe 607 can be positioned at a depth in the bioreactor well sufficient to maintain submersion of the light pipe 607 in the contents of the well during filling, draining, and stirring. In some embodiments, peripheral sensors can be used that provide a warning if fluid levels in the well drop below the OD sensor light pipe such that the light pipe is no longer fully submerged and, therefore, no longer providing accurate measurements. In an embodiment, the OD sensor can be calibrated to recognize sensor readings that are outside the range of normal operation, which can be the result of the absence of media.

In an embodiment, the OD sensor light pipe 607 can have 45-degree internal reflection at the end of the light pipe that extends into the well. In an example, this configuration facilitates co-location of the OD sensor's optical emitter and photodetector (in contrast to configurations in which the optical emitter and the photodetector are disposed on opposite sides of the well 201).

In some embodiments, an optical emitter and a corresponding photodetector for OD sensing can be placed on opposite sides of a well. In such embodiments, the emitter and photodetector (and any associated light pipes) can be disposed outside the well 201. Alternatively, the associated light pipe(s) can penetrate through the side(s) of the bioreactor well.

### Temperature Sensor

Referring to FIG. 2, each bioreactor unit 200 may include a temperature sensor 261. A temperature sensor 261 can be a non-contact infrared-based sensor that provides for the non-invasive measurement of the temperature of a well 201. Non-contact infrared temperature sensors absorb ambient infrared (IR) radiation emitted by a heated surface (e.g., an outside wall of a well 201), which is suitable for applications where more invasive methods of temperature measurement are either not possible or not desirable. With non-contact infrared temperature sensors, infrared light emitted from a surface of the well and detected by the sensor is converted to an electric signal that corresponds to the temperature of the well's surface. In some embodiments, the measured temperature of the well's surface and the temperature of the well's contents (e.g., growth media) are assumed to be substantially equal. In some embodiments, the temperature sensor can be calibrated for any expected difference between the temperature of the well's surface and the temperature of the well's contents.

In one example, an IR temperature sensor that has a measurement range between 0-50 degrees Celsius can be used. While the normal growth temperature for an organism may not be particularly close to 0 or 50 degrees Celsius, the contents of a well can be cooled to temperatures near 0 degrees Celsius or heated to temperatures near 50 degrees Celsius in accordance with temperature-based selection pressures. In some embodiments, an IR temperature sensor with another measurement range can be used.

In some embodiments, a portion of the temperature sensor (e.g., a light pipe) can be embedded into the molded bioreactor unit 200, such that the temperature sensor is not disposable. Light emitted from an outer surface of the well can then be collected by the embedded temperature sensor light pipe for measuring the temperature of the well 201.

In some embodiments, in addition to monitoring the temperature of the well (or the growing organisms inside the well), the temperature sensor for each bioreactor unit can be used to monitor the temperatures of circuit boards or other bioreactor components for possible malfunction. For example, a circuit board that is too hot could indicate a component failure. A failure in the heating or cooling system can also be detected with unusual temperature offsets. Accordingly, in some embodiments, a certain alert signal can be generated when the temperatures of the monitored boards and/or other components are determined to be outside their expected ranges.

### Fluid Level Sensor

In some embodiments, each bioreactor unit 200 may include a fluid level sensor. In some scenarios, the bioreactor units are expected to run with a constant fluid level during each run. In such scenarios, the fluid level sensor may be optional.

Any suitable type of fluid level sensor may be used, including floater-based level sensors or electrical quantity (e.g., conductivity, resistance, or electrical capacitance)-based level sensors. However, considering the volume of a well 201 within each bioreactor 200 and the large number of bioreactor units supported by the disclosed bioreactor system 100, it may be beneficial to use a capacitor-based level sensor due to its simple design, low cost, and small size. A capacitor-based level sensor can include a parallel-plate capacitor that can be immersed in the liquid. The level change of the liquid can lead to the amount of dielectric material between the plates changing, which causes the capacitance to change as well. The level of the liquid can be calculated based on the sensed capacitance. In some embodiments, an additional pair of capacitive sensors can be used as a reference when calculating the capacitance/liquid level change. In some embodiments, the capacitor-based level sensor can measure the liquid level in a minimally invasive manner as only the probes (e.g., plates) are immersed inside the liquid, and their presence causes little disturbance to the growing organisms during the measurement.

In some embodiments, the level sensor can measure the liquid level in a bioreactor well from time to time (e.g., periodically with a predefined frequency (e.g., once every few hours)). In some embodiments, the level sensor can measure the liquid level in a well on-demand (e.g., based on a request from an operator) or based on certain activities of the bioreactor 200. For example, when a material is dispensed into the well or waste is removed from the well, the level sensor can be triggered to measure the liquid level after such activity. With this approach, the bioreactor unit can confirm whether an expected amount of material was dispersed into the well or an expected amount of waste was removed from the well. Frequent or timely measuring of the liquid level inside a well can also be used to verify that there is sufficient aeration for the growing organism inside the well, or that the liquid level is not too high to block the gas exhaust port or outlet, or that the liquid level is not so low that the bottoms of the pH and dissolved oxygen sensor channels are not submerged in a well's growth media.

In some embodiments, the measured liquid level can be transmitted to the bioreactor controller board, which can be configured to maintain the liquid within a vessel at a predefined level by either instructing the media valve 215c to dispense more media into the well 201 or the waste valve 225 to remove waste from the well 201.

### 1.4 Summary

To allow a better understanding of the structure and operation of the above described individual bioreactor units 200, CAD drawings illustrating embodiments of the bioreactor units are further presented and described.

FIG. 6A illustrates a cross sectional view of a bioreactor unit 600, according to some embodiments. As illustrated in the figure, the bioreactor unit 600 can include two manifolds (e.g., diffusion bonded manifolds), a top manifold 601 and a bottom manifold 603, where the top manifold 601 can be a part of the top manifold for a whole bioreactor cluster, while the bottom manifold 603 can be a part of the bottom manifold for a whole row of bioreactor units, as will be described in greater detail later. The diffusion bonded manifolds may be formed from any suitable material, including (without limitation) thermoplastic or acrylic materials. This split manifold architecture allows the distribution of fluidic channels to each individual bioreactor unit without the need for a large number of tubes and fluidic fittings. Integrating the fluidic channels into the manifolds greatly reduces the risk of leaks and assembly time, and increases the density of the bioreactors so that more can fit in a set space.

As also illustrated in FIG. 6A, between the two manifolds 601 and 603 is a bioreactor well (e.g., an injection molded bioreactor well) 605. An optical density light pipe 607 disposed on one side of the bioreactor well 605 can measure the optical density of the well's contents (e.g., organisms growing inside the well 605). Below the bottom manifold 603, a stir bar motor 609 can drive a stir bar 611 to mix the growth media inside the well 605. The rotatable stir bar 611 can be disposed in any suitable location in the well (e.g., in the center of the bottom portion of the well).

As also illustrated in FIG. 6A, on the right side of the well 605, there is a TEC 613, which can be used to control the temperature of the bioreactor 605. Adjacent to the TEC 613 is a heat spreader (or cold finger) 615, which helps distribute the heating or cooling generated by the TEC 613 around the bioreactor 605.

Also included in the illustrated bioreactor unit are valves 617 for media, an auxiliary substance (aux), and O₂. Although illustrated on the right side of the bioreactor in FIG. 6A, in actual applications, these valves can be disposed behind the illustrated bioreactor (e.g., behind the illustrated plane in FIG. 6A). In some embodiments, the valves 617 are mounted to the underside of the top manifold 601 (i.e., the side of the top manifold that faces the contents of the bioreactor well 605), such that the valves 617 do not interfere with the movement of the gantry head above the bioreactor units. In the lower part of FIG. 6A, a waste valve 619 can be operated to remove waste from the well 605. Also included in the illustrated bioreactor unit is a bioreactor printed circuit board (PCB) 621, on which various components may be mounted, e.g., the optical density sensor 607, temperature sensor, communication units, and other circuits or devices for manipulation, control, and/or sensing of the bioreactor unit.

FIG. 6B illustrates another view of a bioreactor unit 620, according to some embodiments. From the figure, it can be seen that the heat spreader 615 surrounds the bioreactor with a ring structure so that heating or cooling generated by the TEC 613 can be distributed around the bioreactor well 605.

FIG. 6C illustrates another view of a bioreactor unit 640, according to some embodiments. As illustrated, the top manifold 601 can be a contiguous structure to which all the bioreactor units in a cluster are mechanically and fluidically coupled, while the bottom manifold 603 can be segmented into distinct structures that are mechanically and fluidically coupled to the bioreactor units in a row. The different segments of the bottom manifold 603 can be separated by bioreactor PCB boards 621, each of which can control the operation of the bioreactor units in an adjacent row. As described above, optical density sensors, communication units, and certain other circuits or devices may be mounted to the bioreactor PCB board.

In the example of FIG. 6C, TEC 613 is adjacent to the heat spreader 615, which surrounds the bioreactor well 605. As can be also seen in FIG. 6C, the three valves for aux, media, and O₂ can be disposed on the top manifold 601 and connected to the fluidic channels for these materials. The fluid inlets for the aux, media, and O₂ can be positioned close to the top of the bioreactor well 605, and aligned together at a same height. Also illustrated in FIG. 6C is a part of a stir bar motor 609 and a valve 619 for controlling waste removal, where the fluidic channel for the waste removal is molded into the bottom manifold.

The illustrated bioreactor units in FIGS. 6A-6C are for illustrative purposes, but not for limitation. An actual bioreactor unit can include more or fewer components than those illustrated in FIGS. 6A-6C. For example, the lid for a bioreactor is not included in FIGS. 6A-6C. In addition, the arrangement and scale of the components can differ from those illustrated in FIGS. 6A-6C.

In some embodiments, each bioreactor PCB may include one or more processing devices or controllers configured to execute software and/or firmware to control the operation of the corresponding bioreactor units. FIG. 6D illustrates an exemplary software/firmware stack 660 for a bioreactor PCB, according to some embodiments. As illustrated in the figure, the software/firmware for a bioreactor PCB can operate on a real time operating system (RTOS) 661. The RTOS can maintain an internal counter synchronized to a clock in a cluster controller (e.g., on the cluster PCB) that controls a whole bioreactor cluster. The RTOS can ensure that bioreactor operations occur in a tightly coordinated fashion.

In some embodiments, the software/firmware stack 660 also includes an application layer 663 of software that can determine what actions to take based on various inputs and initiate those actions (e.g., controlling the valves on a bioreactor's fluidic channels, obtaining a measurement of bioreactor temperature, etc.), and a hardware abstraction layer 665 that interfaces to the hardware platform (e.g., one or more processing devices or controllers) 667. The software/firmware stack 660 can also include an application program interface layer 669 that resides between the hardware abstraction layer 665 and the application layer 663, and drivers 671 that initialize the hardware components of the bioreactor PCB and manage access to the hardware components by higher layers of software, e.g., the RTOS. One of ordinary skill in the art will appreciate that the specific APIs 669 and drivers 671 included in FIG. 6D are for illustrative purposes only and not for limitation. In some embodiments, depending on the components included in a bioreactor unit, other APIs 669 and/or drivers 671 can be used.

### 2. GROUPING OF BIOREACTOR UNITS

As described earlier, in the disclosed bioreactor system 100, the individual bioreactor units are grouped together as bioreactor clusters. That is, a bioreactor cluster is a modular unit inside the system 100. Each cluster can include a predefined number of individual bioreactor units, which may be organized in a 2D array. For example, each bioreactor cluster can include a fixed number (e.g., 64) of bioreactors in a fixed number (e.g., 8) of rows. In some embodiments, each row of bioreactor units can be grouped together to share certain components. Grouping together bioreactor units into rows and further into clusters can alleviate some key obstacles to the production and use of large-scale bioreactor systems, including but not limited to: 1) improving the reliability of the individual bioreactors, and 2) reducing the costs of production by sharing sensors and other accessories among a large number of individual bioreactors.

For example, by integrating (e.g., molding) fluidic channels (211, 221) for aux, media, O₂, and waste into the top and bottom manifolds, “last mile” plumbing for each bioreactor can be mass produced in a cost-effective and reliable manner. That is, instead of trying to connect hundreds or thousands of individual pieces of tubing, the fluidic channels can be integrated (e.g., molded) into the manifolds, thereby greatly improving reliability and reducing component count and production cost.

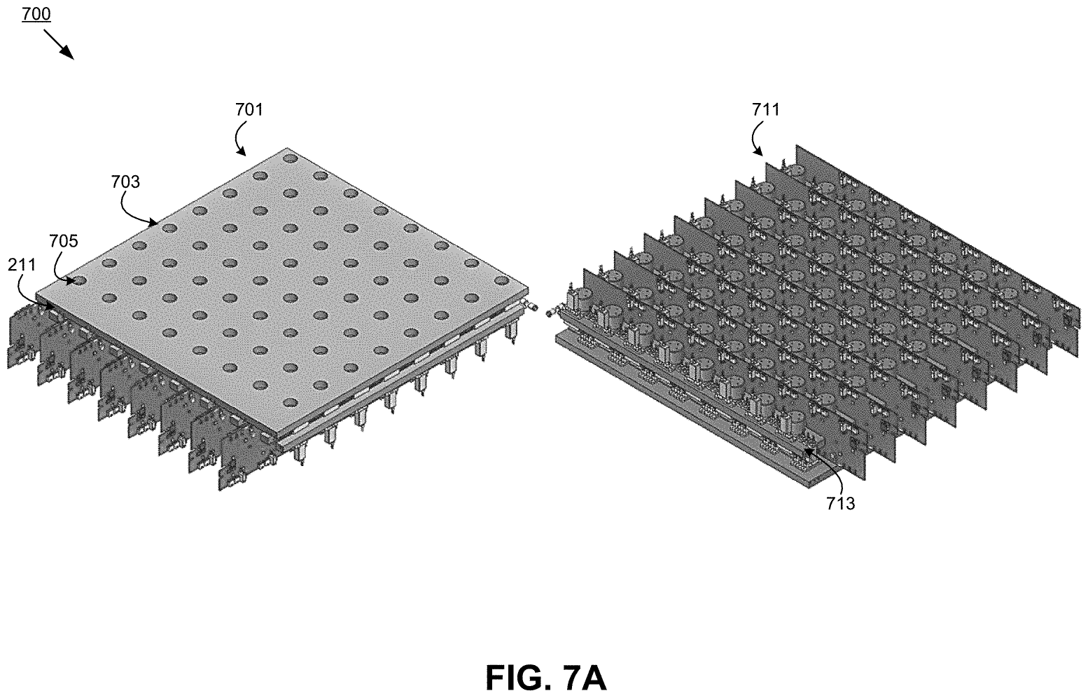

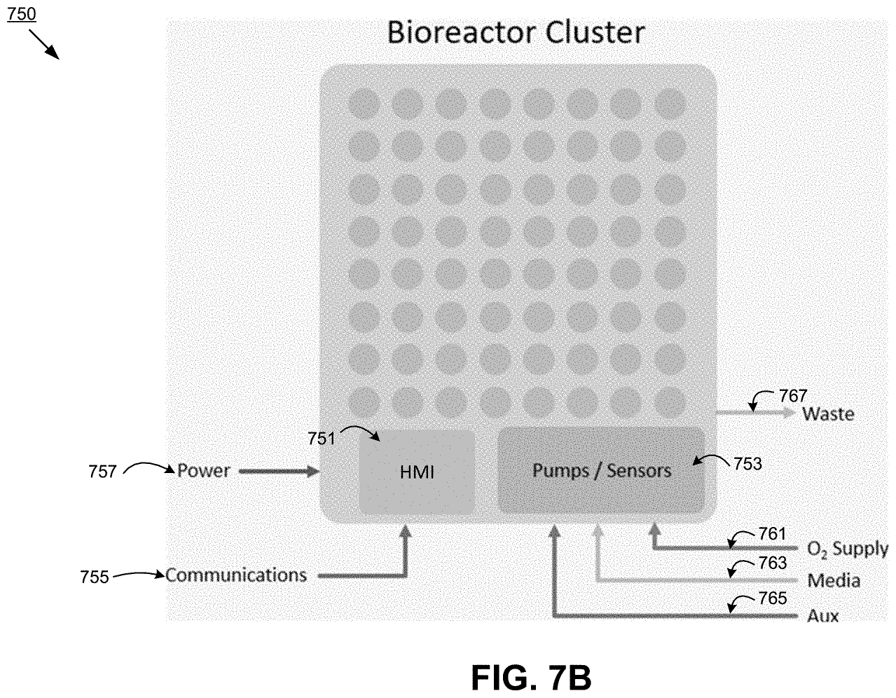

Referring to FIG. 7A, a top view 701 and bottom view 711 of an example bioreactor cluster 700 are illustrated. As illustrated in the top view 701, a bioreactor cluster 700 can group together a predefined number of bioreactor units in an array, e.g. 64 bioreactor units in a 2D, 8×8 array. As can be also seen in the top view 701, the top manifold 703 can be a single-piece manifold that is mechanically and fluidically coupled to all the bioreactor units in the cluster. The single-piece top manifold 703 can be formed through a diffusion bonding process. The fluid channels 211 that provide material (e.g., O₂, media, and aux) to the individual bioreactor units can be formed during this process and included in the top manifold 703. Each bioreactor well 705 can be also formed through a similar injection molding process, and then inserted into the pre-formed top manifold 703 that includes corresponding openings for insertion of these bioreactor wells 705.

From the bottom view 711 of the cluster 700 in FIG. 7A, it can be seen that adjacent rows of bioreactor units are separated from each other by the bioreactor PCBs below the top manifold. As can be also seen in FIG. 7A, each row of bioreactor units can include a bottom manifold 713, which can be formed through a diffusion bonding process. In some embodiments, the bottom manifold 713 for a row of bioreactor units is a single, contiguous structure shaped to leave space for other accessory components of the bioreactor units, as shown in FIG. 7A. In other embodiments, the bottom manifold 713 for a row of bioreactor units includes two or more pieces (e.g., two separate manifolds on opposing sides of the row of bioreactor units). The fluid channels 221 for waste removal from the bioreactor units in the row of bioreactors can be formed inside the bottom manifold 713 during the molding process.

In some embodiments, in addition to the bioreactor wells and the associated accessories (e.g., fluid channels, TECs, heat spreaders, light pipes, PCBs, sensors, etc.), a bioreactor cluster can further include a cluster board for controlling and monitoring operations of the bioreactor units in the cluster, as further described in greater detail later.

FIG. 7B illustrates a schematic diagram of a bioreactor cluster 750, according to some embodiments. As illustrated in the figure, in addition to an array of bioreactors, a bioreactor cluster 750 can further include a human-machine interface (HMI) 751, certain pumps and sensors 753, a communication interface 755 for communicating with a master controller of the bioreactor system 100, and a power supply 757 for providing energy for driving the operation of the bioreactor cluster.

The HMI 751 can be used for locally monitoring the cluster 750. As will be described later, each bioreactor unit in the disclosed bioreactor system 100 can self-configure and communicatively connect to the master controller during the initialization of the bioreactor system 100 or during each run (e.g., at the beginning of each run). The HMI 751 included in the cluster 750 can provide a prominent visual indicator to signal whether the cluster 750 has lost connection or failed to connect with the master controller. The inclusion of the HMI 751 thus simplifies the setup of the disclosed bioreactor system, while also facilitating the convenient monitoring of the operations of each bioreactor cluster and/or tracking problems that can arise within each cluster.

The sensors and pumps 753 can be configured to control the ingress of fluids into the bioreactor cluster 750 and/or the egress of waste from the bioreactor cluster 750. As previously described, each bioreactor cluster includes a large number of fluid channels to provide substances to the individual bioreactors. These fluid channels 211, which are formed in the top manifold and connected (e.g. fluidically coupled) to the individual bioreactors in the cluster, can be further connected (e.g., fluidically coupled) to the corresponding resource supply channels of each bioreactor cluster, such as O₂ supply channel 761, media supply channel 763, and aux supply channel 765. Each source supply channel can have a flow sensor and a pump (e.g., peristaltic pump) to ensure consistent and precise delivery of the fluids to the individual bioreactors in the cluster.

Likewise, as previously described, each bioreactor row includes waste channels 221 to remove waste from the individual bioreactors. These waste channels 221, which are formed in the bottom manifold and connected (e.g. fluidically coupled) to the individual bioreactors in the row, can be further connected (e.g., fluidically coupled) to the corresponding waste removal channel 767 of the bioreactor cluster. The waste removal channel 767 can have a flow sensor and a pump (e.g., peristaltic pump) to ensure consistent and precise removal of waste from the individual bioreactors in the cluster.

The communication interface 755 can initialize and manage communications with the master controller of the bioreactor system 100. Such communication can include transmitting data (e.g., OD values indicating growth of organisms inside bioreactor units) collected from the sensors associated with the individual bioreactor units to the master controller and/or another data processing or storage unit of the disclosed bioreactor system 100, and/or receiving commands (e.g., commands for instructing selection pressures to be applied to specific bioreactor units) from the master controller to the bioreactor units.

The power supply 757 can provide the electrical energy for driving the operations of the components inside the bioreactor cluster 757. For example, all the TECs, the pumps, the HMI, and the optical sources for sensors included in the bioreactor cluster may consume electrical power during operation.

Power analysis can be performed to determine the expected average and/or peak power used by each individual bioreactor unit, each bioreactor cluster, each station, and the whole bioreactor system 100. Due to the large number of bioreactor units included in the bioreactor system 100, the power used by the whole system 100 can be significant. For example, if the peak power used by an individual bioreactor unit is 24 W, and a total of 10,000 bioreactor units are included in the bioreactor system 100, the peak power used by the bioreactor system 100 can be quite large, which can potentially place significant constraints on the design of the system's power supply. In some embodiments, software executed by the master controller or the cluster boards can be configured to stagger operations of the bioreactor units in the system 100, to avoid simultaneously running all of the bioreactors at the peak power. This technique can significantly lower the peak power drawn by the bioreactor system. In some embodiments, by controlling the ambient environmental temperature around the bioreactor system to be close to an optimal temperature for the growing organisms, the power consumption for each bioreactor unit can be further decreased.

In some embodiments, a bioreactor cluster 750 can include additional components not illustrated in FIG. 7B, such as communication interfaces for communication with certain gantry components or with other bioreactor clusters, as will be described in detail later. In the following subsections, aspects of bioreactor clusters and rows are described in greater detail, as well as groupings of two or more clusters and additional functionality associated with such groupings.

### 2.1 Rows of Wells

As described earlier, the individual bioreactor units in a bioreactor cluster can be organized into rows, where each row can be integrated together by sharing a common bottom manifold and a bioreactor PCB. The shared bioreactor PCB can be a single board positioned between two adjacent rows of bioreactor units (except one board located on the edge of the bioreactor cluster), and can allow mounting of accessory components of each bioreactor unit, such as light pipes and certain optical sources, as can be seen from FIG. 7A. The shared bottom manifold can hold individual bioreactor units together in a row with fluid channels (e.g., waste removal channels) connected to each bioreactor well embedded therein.

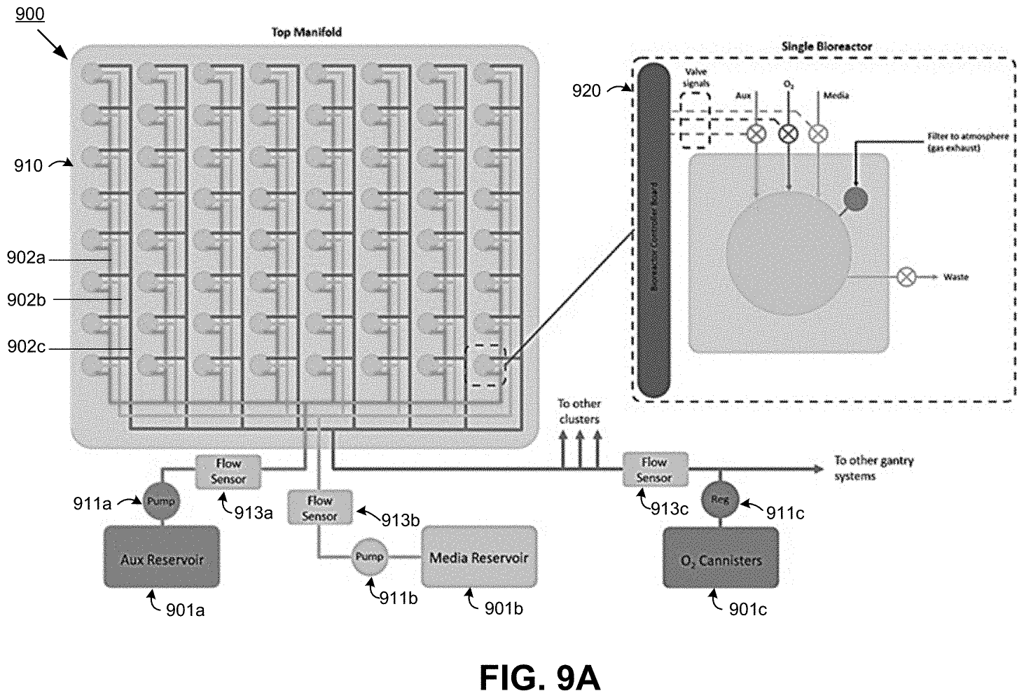

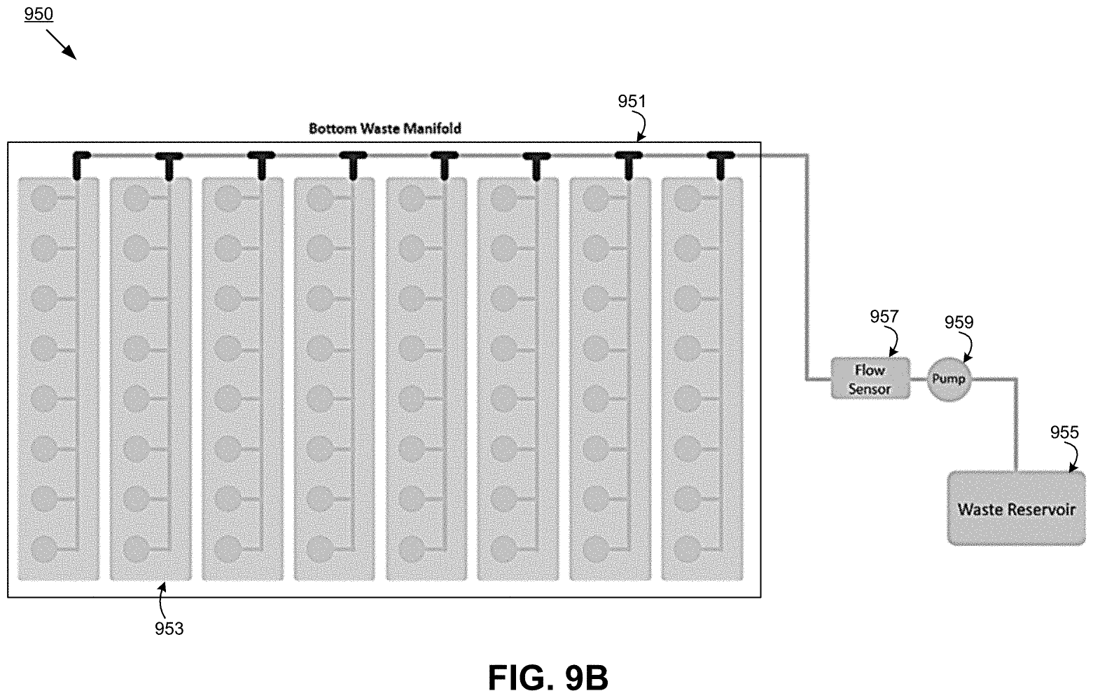

In some embodiments, the shared bottom manifold can be a diffusion bonded fluidic manifold manufactured through a diffusion bonding process. Diffusion bonding is an advantageous technique for manufacturing a plastic fluidic manifold, with leak-proof, sealed tracks and chambers between layers without the use of any adhesives or sealing gaskets. Diffusion bonding facilitates creation of smooth, curved channels, interfacing over several layers to produce complex, multi-channel fluidic manifolds. For example, referring to FIG. 9A, for fluid channels in the portion of the top manifold corresponding to a row of bioreactor units, a main flow channel 902 for each source (e.g., aux, media, and O₂) can be shared by all the bioreactor units in the row. Still referring to FIG. 9A, sub-channels for each fluid can branch from the respective main flow channel and extend into each bioreactor well. This kind of channel complexity is feasible with the diffusion bonding process. Diffusion bonded plastic manifolds have certain other properties that make them advantageous for manufacturing top and bottom manifolds in the disclosed large-scale bioreactor system 100. For example, using diffusion bonded fluidic manifolds in the bioreactor system 100 can reduce assembly time and costs, improve reliability, reduce component count and costs, significantly reduce potential leak points, eliminate the potential for incorrect connections in assembly and serving, reduce dead volume and the requirements to plug channels, and can allow visualizing of system operation and optical sample analysis.

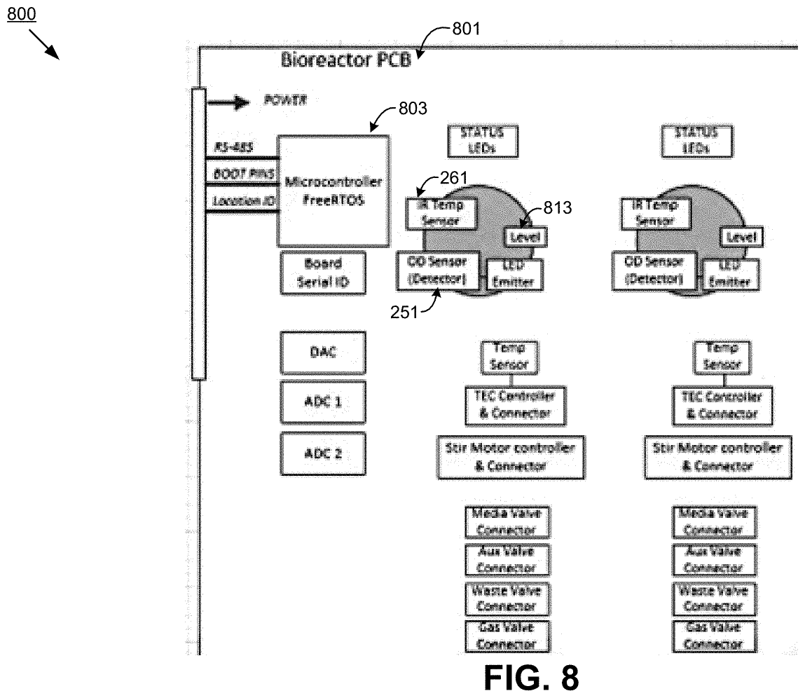

In some embodiments, in addition to the main flow channels 902, each row of bioreactor units can further share a bioreactor board that manages communications between the bioreactor units in the row and the cluster controller board. FIG. 8 illustrates one exemplary configuration of a bioreactor PCB 801 for each row of bioreactor units. According to some embodiments, the cluster controller board for each bioreactor cluster can have a communication interface for communicating with a set of bioreactor boards (e.g., 8 bioreactor boards for 8 rows of bioreactor units) in the cluster. In some embodiments, each bioreactor board 801 in a bioreactor cluster can be swapped out with another board of the same type, to simplify maintenance.

In some embodiments, each bioreactor board can include (e.g., store) a unique serial number that can be used to identify the board and provide a unique ID for each bioreactor unit controlled by the board. The interface between the bioreactor boards and the cluster controller board can identify the locations of the bioreactor boards in the bioreactor cluster. Based on these unique IDs and location information, each sensor reading from the shared gantry can be correlated with the individual bioreactor from which the sensor reading is collected.

As described earlier, each bioreactor board can control all bioreactor units in the corresponding row. For example, 8 bioreactor units in a row can be controlled by the bioreactor board for that row (although only 2 of the 8 bioreactor units are illustrated in FIG. 8).

In some embodiments, a bioreactor board 801 includes a microcontroller 803 that communicates with the cluster controller board (not shown in FIG. 8). As described earlier, each bioreactor unit can include an OD sensor 251, a temperature sensor 261, and an optional liquid level sensor 271. The OD sensor 251 can be used to assess and control system performance. The temperature sensor 261 can be a non-contact IR-based sensor for temperature measurement. The liquid level sensor 271 can be used to assess and control liquid levels in a bioreactor well. In some embodiments, a portion of the liquid level sensor may be disposed within the well 201, and the circuit 813 that reads the liquid level measurements may be disposed on the bioreactor board 801. Circuitry and support electronics are also provided for the valves (e.g., media valve, aux valve, waste valve, gas exhaust valve), the TECs, and the mixing systems (e.g., stir bar motor) for respective bioreactor units included in each row. The bioreactor board 801 may control the operation of each component, based on the commands received from the cluster controller board. For example, the cluster controller board may send a command to a bioreactor board to instruct the mixing system to rotate the stir bar at a speed of 800 RPM. For another example, the cluster controller board may send another command to a bioreactor to instruct a certain amount of media to be added to a bioreactor well.

### 2.2 Clusters

As described earlier, the individual rows of bioreactor units can be grouped together to form a bioreactor cluster, in which the individual bioreactor units may be organized in a 2D array. As described earlier with reference to FIG. 7A, a bioreactor cluster can include a top manifold shared by all bioreactor units in the cluster and respective bottom manifolds for each row of bioreactor units included in the cluster.

FIG. 9A illustrates a schematic diagram of fluid channels and pumping system 900 for a top manifold 910 of a bioreactor cluster, according to some embodiments. As illustrated in the figure, the bioreactor units in the bioreactor cluster share single sources of aux, media, and O₂ (e.g., aux reservoir 901a, media reservoir 901b, O₂ canisters 901c). Each of the three sources has a corresponding pump (e.g., pumps 911a and 911b for aux liquid flow control and media liquid flow control, respectively) or regulator (e.g., regulator 911c for O₂ gas flow control) and a corresponding flow sensor (e.g., flow sensors 913a, 913b, 913c) to control and monitor the supply of the respective material to the bioreactor cluster. Each fluid source 901 can be distributed to each row of bioreactor units through a shared fluid channel 902, which then further distributes the source to the individual bioreactor units through the corresponding fluid sub-channels branched from the shared channel. Each bioreactor unit has corresponding valves (e.g., binary valves) for controlling the dispensing of the source materials to the bioreactor well, as can be seen from the enlarged schematic for an individual bioreactor unit 920 in FIG. 9A. The valves of the bioreactor unit can be controlled to open or close based on the valve signals received from the unit's bioreactor controller board, which further receives signals from the cluster controller board of the bioreactor cluster and/or further from the master controller of the whole bioreactor system. For example, the master controller can determine a selection pressure to be applied to each bioreactor unit, e.g., a certain pH value, a certain concentration of DO, or a certain concentration of growth media for each bioreactor, and can send commands to these bioreactor units as valve signals (e.g., through corresponding cluster controller board and bioreactor controller board) to control the dispensing of the corresponding amount of materials. FIG. 9B further illustrates a schematic diagram of fluid channels and pumping system 950 for bottom manifolds of a bioreactor cluster, according to some embodiments. As can be seen, for a single bioreactor cluster 951, each row of bioreactor units has a respective bottom manifold 953. The fluid channels for the bioreactor units in each row are molded into the respective bottom manifold. Removed waste can flow from each bioreactor unit into the waste reservoir 955 based on the control of the flow sensor 957 and the corresponding pump 959. In some embodiments, the pump 959 is a vacuum pump that allows a bioreactor to remain isolated and prevents cross contamination.

In some embodiments, the specific rates of removing waste from bioreactor units are controlled by a cluster controller board for the bioreactor cluster, similar to the dispensing of the materials into bioreactor wells.

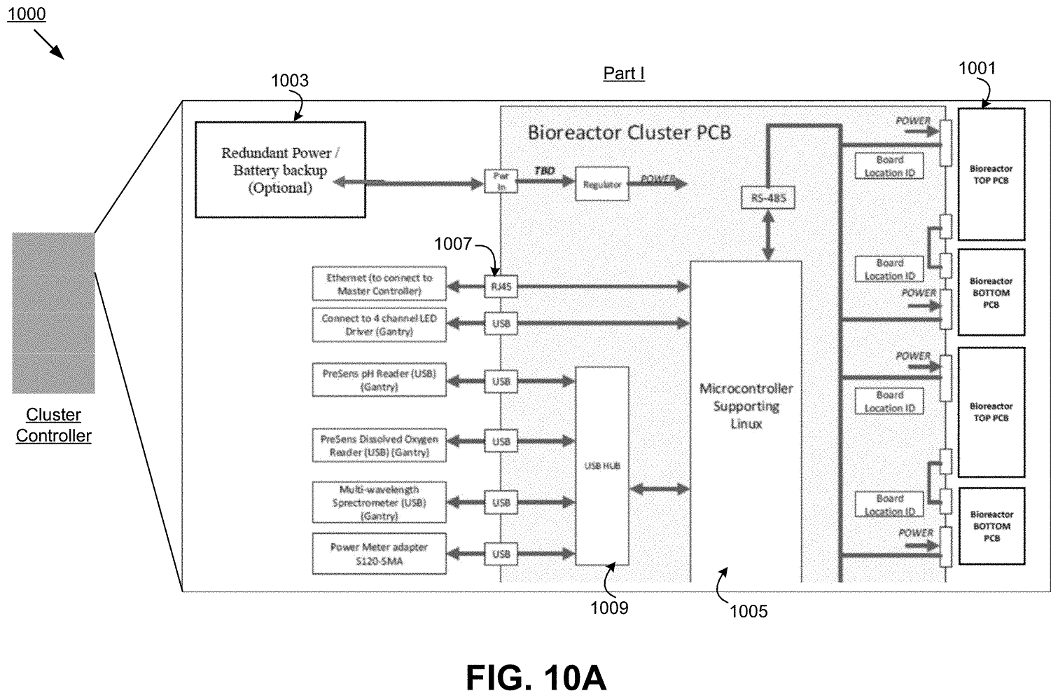

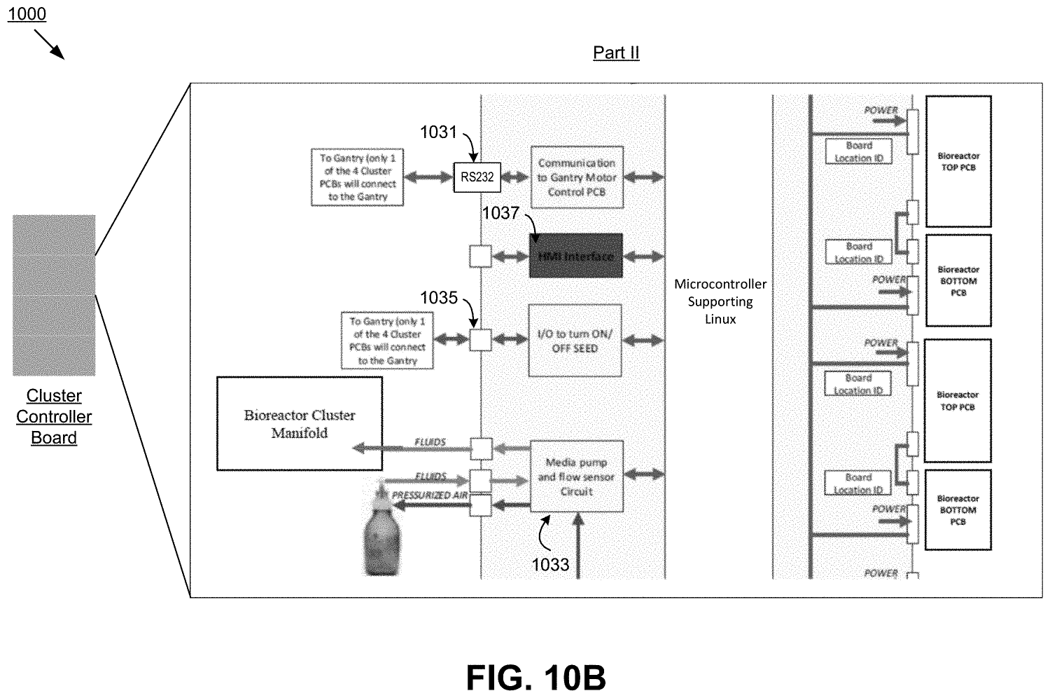

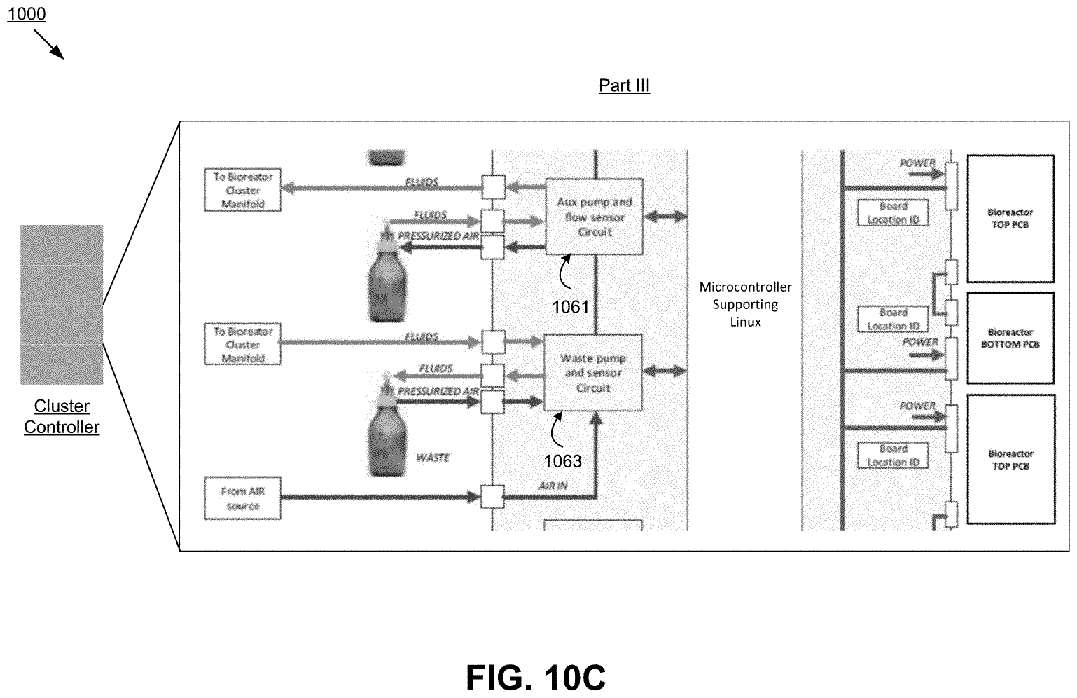

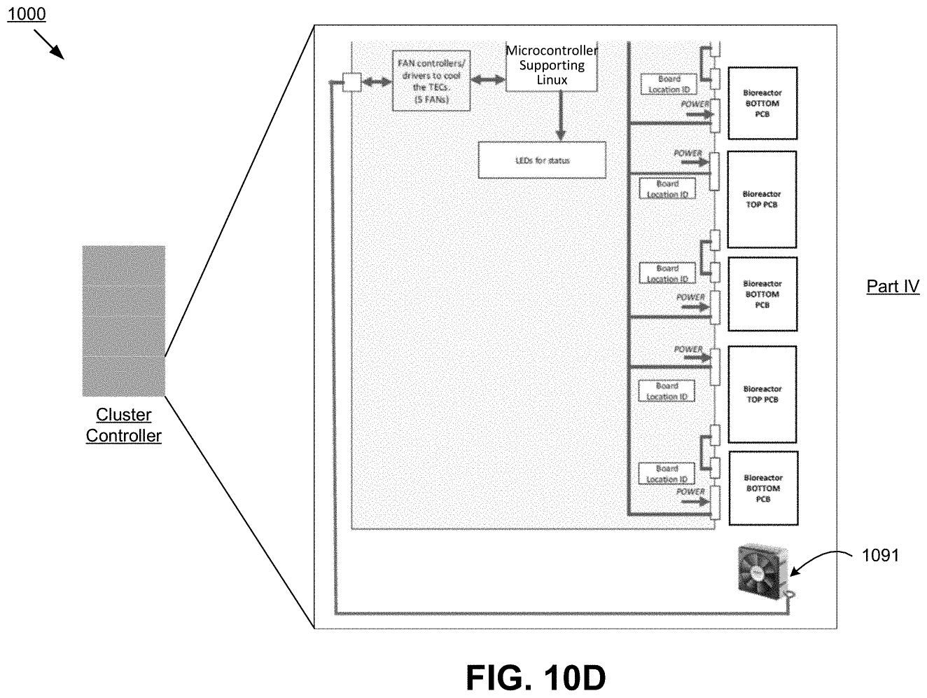

FIG. 10A-10D collaboratively illustrates a schematic diagram of a cluster controller board 1000 of a bioreactor cluster, according to some embodiments. In some embodiments, the cluster controller board can manage the gantry, the gantry sensors, the pumps and corresponding flow meters, and the bioreactor boards. Each cluster can include one cluster controller board and a number of bioreactor circuit boards (e.g., one bioreactor circuit board per row).

As illustrated in Part I of FIG. 10A, a cluster controller PCB can distribute electric power to all other components in the bioreactor cluster. For example, the cluster controller PCB can distribute power to each bioreactor board 1001. Although there are two separate bioreactor boards illustrated for each row of bioreactors in FIG. 10A (a top PCB and a bottom PCB), in some embodiments, there can be a single bioreactor board for each row of bioreactor units. In some embodiments, the cluster controller may provide power to other components through USB connection. For example, as illustrated in FIG. 10A, there can be a USB hub 1009, which is configured to supply power to different components included in the bioreactor cluster through USB connection. For example, the USB hub can provide power to LED drivers, pH readers, DO readers, multi-wave spectrometers, and meter adapters included in the bioreactor cluster, as shown in FIG. 10A. In some embodiments, the power supply for a bioreactor cluster can have dual power inputs (e.g., redundant power/battery backup 1003) to prevent losing a bioreactor cluster during a run due to power supply failure.

In some embodiments, the cluster controller board can connect to (e.g., communicate with) the master controller of the disclosed bioreactor system 100 via Ethernet. For example, a microcontroller 1005 included in the cluster controller board may communicate with the master controller through the communication bus RJ45 (indicated by 1007 in FIG. 10A).

In some embodiments, to ensure the heating and cooling system can maintain proper functioning, the bioreactor cluster PCB can control fans included in the bioreactor system for removing heat (e.g., heat generated by the TECs). One such fan 1091 is illustrated in FIG. 10D. It is to be noted that in actual application, multiple fans may be included in the disclosed bioreactor system 100 and these fans may be evenly distributed around the disclosed bioreactor system for better removal of heat (e.g., heat generated by the TECs).

In some embodiments, each cluster controller board can connect to (e.g., communicate with) several bioreactor boards (e.g., the bioreactor boards for all rows in the cluster). For example, there can be a communication system between all the bioreactor boards in the cluster and the cluster controller board. An example of such a communication bus is RS485, as shown in FIG. 10A. In some embodiments, the average data rate that the bus supports can be up to 2 KB/s (120 KB/minute) or any other suitable value. In some embodiments, each bioreactor unit produces less than 1 KB of data per minute on average, which translates to 64 KB/minute in a cluster with 64 bioreactor units. There can be some overhead as there may be other information passed from the cluster controller to bioreactor boards. In some embodiments, if there are two bioreactor controller boards (e.g., top and bottom boards) for each row of bioreactor units, there is a separate communication bus between each pair of boards, to avoid the latency of using the global bus shared between all the bioreactor boards.

In some embodiments, each bioreactor board can identify its physical location within the bioreactor cluster by using a board location ID provided by a bioreactor cluster PCB. This means each board can self-configure upon power-up.

In some embodiments, the cluster controller board can connect to a gantry associated with the bioreactor cluster. For example, as shown in FIG. 10B, the bioreactor cluster board may communicate with the gantry through a communication bus RS232 or a different bus, as indicated by block 1031 in the figure. The signals communicated between the cluster controller board (or the microcontroller 1005) and the gantry may include motor drive signals for the X, Y, and Z positions of the gantry head. These signals may direct the gantry to move according to a certain pattern. For example, the cluster controller may direct the gantry to seed each bioreactor unit one-by-one, row-by-row, and/or cluster-by-cluster when one gantry covers multiple bioreactor clusters. In addition, the gantry may send position signals to the cluster PCB indicating the position of the gantry head on each axis.

In some embodiments, depending on the configuration of the gantry, an additional controller board can be included in the gantry for connection to the cluster controller to drive the gantry. The gantry controller board can have integrated controllers and stepper motor drivers for driving and controlling the motion of the gantry.

In some embodiments, the gantry sensors can be controlled by the cluster controller board and/or the gantry controller board. For example, when the gantry is correctly aligned with a bioreactor unit, the cluster controller board can activate light sources and read out the sensor values from the gantry sensors. For example, the pH value for a bioreactor unit may be read out by the pH sensor activated by the cluster controller board.

In some embodiments, the cluster controller board can monitor and control the seeding process performed by the gantry. For example, the cluster controller board can have a communication interface 1035 for sending commands to instruct the gantry to seed bioreactors in the cluster, as illustrated in FIG. 10B. In some embodiments, the instruction may merely instruct the gantry to start the seeing process. The gantry controller board may be configured to perform a predefined seeding procedure in which the gantry seeds the bioreactor units one-by-one, row-by-row, and cluster-by-cluster. In some embodiments, the gantry can provide each bioreactor unit with the same amount of seeding solution. In some embodiments, the instructions can instruct the gantry to seed a specific bioreactor unit.

In some embodiments, the cluster controller board can control the inlet and outlet pumps, including the pumps for dispensing materials into wells and the pump for removing waste from the bioreactor units. In some embodiments, the cluster controller board can monitor the flow sensors, and feed this information back to the bioreactor boards and to the master controller. Accordingly, the cluster controller board can include media pump and flow sensor circuit 1033, aux pump and flow sensor circuit 1061, waste pump and sensor circuit 1063, and O₂ flow sensor circuit (not shown in FIGS. 10A-10D), as shown in FIGS. 10B and 10C.

In some embodiments, the cluster controller board can include an HMI interface 1037, as illustrated in FIG. 10B. The HMI interface can be used for interacting locally with the associated bioreactor cluster. The HMI interface 1037 can support at least two functions. The first function is to provide a visual indication of the status of the cluster. The second function of the HMI interface 1037 is to allow a user to configure and/or control a cluster.

For example, if the bioreactor cluster has not connected to the master controller, the cluster will have an alert light that a problem has occurred, which can be displayed through the HMI interface. In some embodiments, the master controller can activate a cluster's status light if an operator is trying to find a specific bioreactor cluster. For another example, an operator can connect to the bioreactor cluster and turn on and off valves, check the status of flow sensors and check the operation of the gantry. In some embodiments, the HMI interface 1037 also allows for configuring and checking network status.

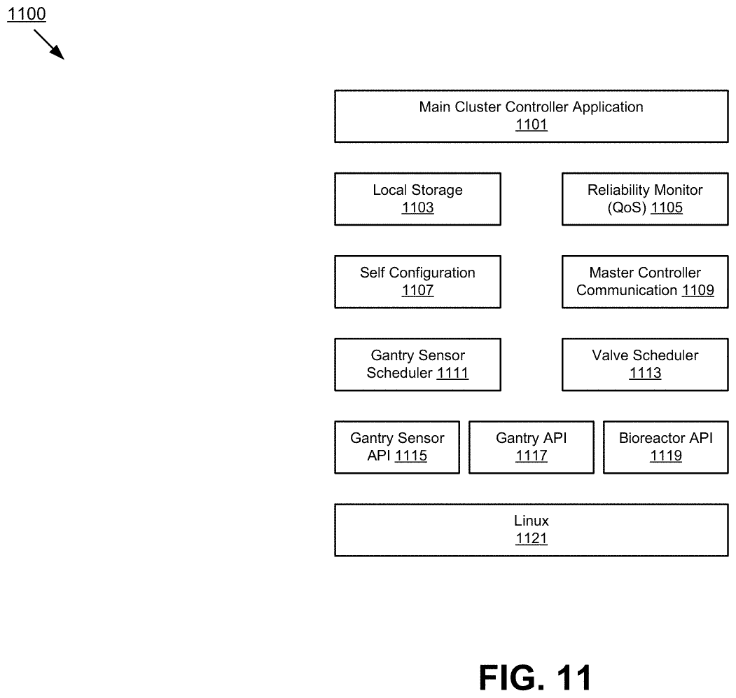

In some embodiments, cluster software 1100 can be specifically configured for the cluster controller to coordinate the activity of all the bioreactors in the cluster and the gantry that services the cluster, as illustrated in FIG. 11.

FIG. 11 illustrates cluster control software 1100 that can be executed by a cluster controller board, according to some embodiments. As illustrated in the figure, the software includes a main cluster controller application 1101 for managing the operation of the cluster, a local storage 1103 for locally storing certain data, a reliability monitor 1105 for checking the status of the cluster, a self-configuration unit 1107 for self-configuration of the cluster, a master controller communication unit 1109 for initializing and managing communications with the master controller, a gantry sensor scheduler 1111 for scheduling the sensors on the gantry (e.g., when to activate the DO sensor, pH sensor, and spectrometer), a valve scheduler 1113 for scheduling the dispensing of materials (e.g., media, aux, and O₂), a gantry sensor unit 1115 for manipulating the sensor operation on the gantry (e.g., controlling optical sources to emit light for sensing), a gantry API 1117 for communicating with the gantry, a bioreactor API 1119 for interacting with bioreactor boards, and a Linux operating system 1121.

In some embodiments, because of the complexity of the cluster control software, the cluster controller board can run Linux 1121, although other software operating systems can be used. A daughterboard with a complete Linux controller can be used to reduce engineering costs, and save on production costs. There are a number of networking protocols the cluster control board can support, including Zero Conf, NTP, and DHCP. These protocols allow the cluster controller and master controller to self-configure the entire system, and maintain a consistent time base across all the components of the system. Communication between the master controller and the cluster controllers can be over TCP/IP15. If communication is lost between the master controller and the cluster controller, the cluster controller can buffer the results in the local storage 1103 until the master controller comes back online.

In some embodiments, to facilitate precise dispensing of source materials, the cluster controller board can maintain a scheduling system for each bioreactor. Opening multiple valves on the same channel at the same time would make it difficult to calculate how much fluid is dispensed into each bioreactor via the shared channels, because the resistance in the channel is heavily dependent on the distance from the pump. Thus, by having the cluster controller board schedule when a bioreactor can use a channel, a known amount of source material can be delivered to the bioreactor.

The cluster control software 1100 can manage the monitoring and reliability of the entire system. By aggregating all the sensor information for the bioreactor cluster, the software can detect deviations from normal system behavior.

In some embodiments, the cluster controller software, in conjunction with the HMI, allows operators to set up and attach the inputs and outputs to the bioreactor cluster. Via the HMI, an operator can start and stop the pumps, check the flow sensors, and open valves for each bioreactor.

The above described functions of the cluster controller board are provided for illustrative purposes and not for limitation. A cluster controller board may have more or fewer functions than those described above. In addition, in some embodiments, instead of sharing a gantry among multiple clusters, each cluster can have a dedicated gantry. The use of a dedicated gantry for each cluster can save time for measuring signals from the cluster, which can facilitate experiments involving actively growing organisms that benefit from more frequent monitoring of the growth at a certain stage.

### 2.3. Groups of Clusters

As illustrated with FIG. 1, a number of bioreactor clusters (e.g., 4 clusters including 4×64 bioreactor units) can be grouped together and share a gantry system (or simply “gantry”). As described earlier, the two main functions of a gantry are: 1) to deliver the seed to the bioreactors at the start of an experiment, and 2) to collect pH, dissolved oxygen and spectrometer measurements from time to time (e.g., at a predefined frequency (e.g., once every hour) or on-demand) using the gantry sensors. The inclusion of a gantry can allow for the sharing of sensors (e.g., expensive sensors) across multiple bioreactor units. In some embodiments, as further cost savings, the seed, e.g., a bioreactor organism starting matter, can be dispensed by the gantry, as well.

In some embodiments, not every bioreactor cluster in a bioreactor group can control the operation of the shared gantry. Instead, one bioreactor cluster within the group can act as the master cluster to control the gantry. All other bioreactor clusters within the group can communicate with the master bioreactor cluster, or more specifically communicate with the cluster controller board of the master bioreactor cluster. In some embodiments, a standard or off-the-shelf control board can be used to control the gantry.

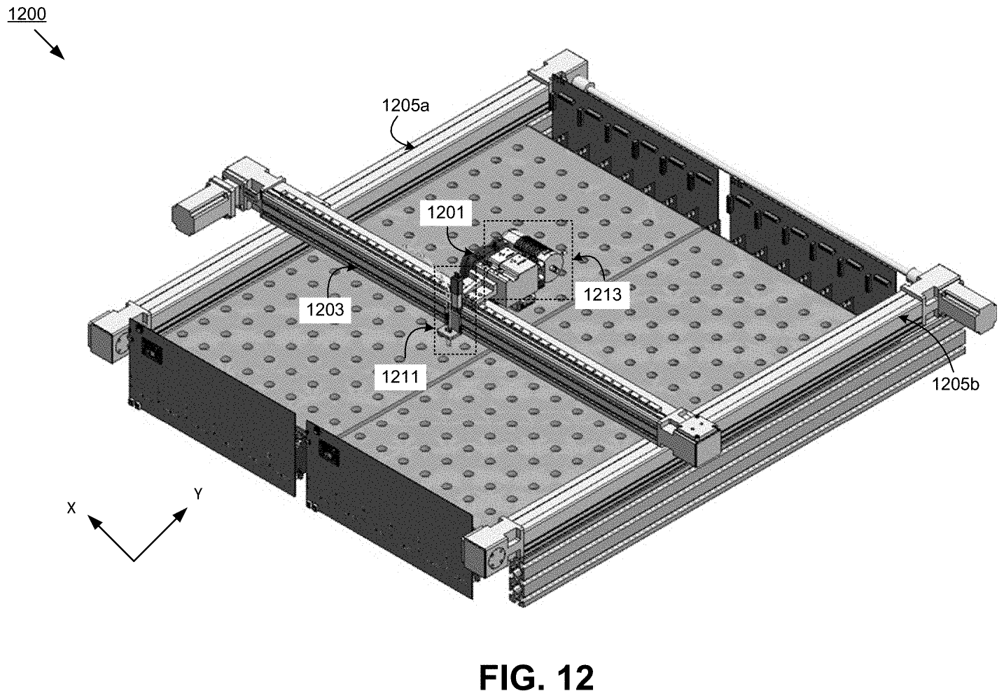

FIG. 12 illustrates a schematic diagram of a gantry system 1200, according to some embodiments. In some embodiments, a Cartesian style XY-gantry can be used to position the gantry head 1201 including sensor probes and seeding apparatus over individual bioreactor units. As illustrated in FIG. 12, the gantry system can include two Y-axis linear guide rails 1205a and 1205b with support hardware, as well as an X-axis guide rail 1203 to which the gantry head is attached. A Z-axis actuator can be included to position the gantry head vertically relative to a bioreactor unit.

In some embodiments, the gantry head 1201 includes a sensor probe unit 1211, which can include the DO sensor probe, pH sensor probe, and spectrometer probe. In some embodiments, the gantry head also includes a sensor pod 1213, which can include the DO sensor (e.g., optical DO sensor), pH sensor (e.g., optical pH sensor), and spectrometer. (Alternatively, in some embodiments, the DO sensor, pH sensor, and/or spectrometer may be disposed on a stationary portion of the gantry rather than the gantry head, and communicatively coupled to the sensor probe unit 1211, which can reduce the amount of power used to move the gantry head.) Collectively, the sensor probes and sensors are suitable for collecting DO measurements, pH measurements, and spectrometer measurements from bioreactor units. In some embodiments, the gantry head 1201 includes a Z-axis actuator for moving the gantry head along the Z-axis (which is perpendicular to the X-axis and Y-axis in the 3D environment) for “dipping” the sensor probe unit 1211 and/or the seeding apparatus into the respective bioreactor units. For example, after the gantry head 1201 is moved to a target position corresponding to the target bioreactor unit, the Z-axis actuator can move the gantry head 1201 along the Z-axis to dip the sensor probe unit 1211 into the lid that includes channels/ports for insertion of probes included in the sensor probe unit 1211. For another example, the Z-axis actuator can move the gantry head 1201 along the Z-axis to inject the seed into a bioreactor unit through a pierceable septum in the lid, which is pierced using a syringe mounted on the gantry head for seeding.

Each bioreactor lid can include a septum for allowing the syringe to pass through the lid to inject the seeding solution or media from the gantry head into a well. In some embodiments, the septum is resealable so that the well is resealed after the seeding, e.g., after the syringe is withdrawn from the septum in the lid. The resealable septum can be easy to penetrate but can remain leak-free after repeated punctures in seeding. In some embodiments, the septum is a silicone septum with sufficiently high elasticity such that the septum reseals after the syringe pierces the septum and is withdrawn. The resealable septum can provide resistance to evaporation of the bioreactor well's contents, and can also avoid fragmentation that can contaminate growth media inside a well.

In some embodiments, the motion of the gantry head 1201 along the X-axis and Y-axis can be predefined. For example, the gantry head 1201 can rest at a default position, which can be on the lower corner of the bioreactor cluster in FIG. 12. When the gantry head 1201 is activated to move, e.g., when the gantry is instructed to seed the bioreactor units serviced by the gantry system, the gantry head 1201 can start to move to the first bioreactor unit adjacent to the corner. After seeding the first bioreactor unit, the gantry head 1201 can move to the second bioreactor unit in a same row along the X direction. The gantry head 1201 can repeat the seeding for the bioreactor units in the first row until the gantry head completes the seeding of the bioreactor units in the first row. If there is more than one cluster in a cluster station, the gantry head 1201 can move to the first row of the adjacent bioreactor cluster and start to seed the bioreactors located in the first row of the adjacent bioreactor cluster.

In some embodiments, after completing seeding the bioreactor units in the first row, the gantry head 1201 can be moved to the second row in the Y direction. The gantry head 1201 may start the second row from the bioreactor unit that is adjacent to the previous one that has just completed seeding, and move back along the opposite direction for seeding the first row. The gantry head 1201 can repeat this process row-by-row until completing all the bioreactor units in the bioreactor group. If more than one cluster is included in the group, all clusters can be completed this way.

In some embodiments, the gantry head 1201 can move to a specific bioreactor unit based on the instruction received from the cluster controller board. In some embodiments, the cluster controller board can identify the target bioreactor unit based on the ID of the bioreactor unit, and provide the instruction including the motor drive signals for the X and Y positions of the gantry head 1201, which corresponds to the target bioreactor unit. In some embodiments, the instruction also includes Z position information (seeding and sensing can be in different Z positions) for the gantry head 1201 to direct the gantry head to “dip” into the target bioreactor unit for seeding or for sensing (e.g., collecting the DO, pH, or spectrometer measurements).

The probes included in the sensor probe unit 1211 can include probes for the gantry's DO sensor, pH sensor, and spectrometer. In some embodiments, the sensor probe unit 1211 may include other sensor probes for collecting other measurements, especially when the sensors corresponding to these probes are expensive.

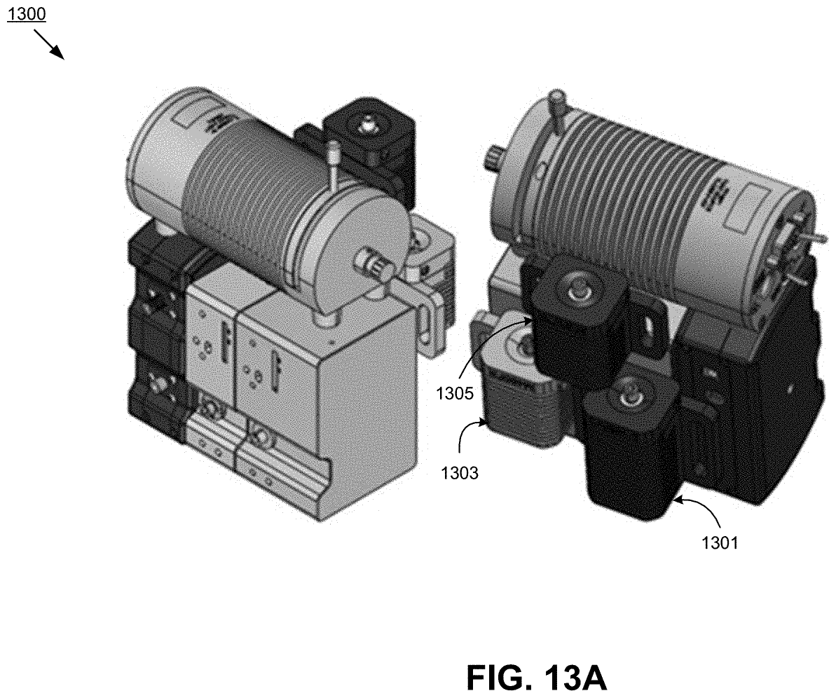

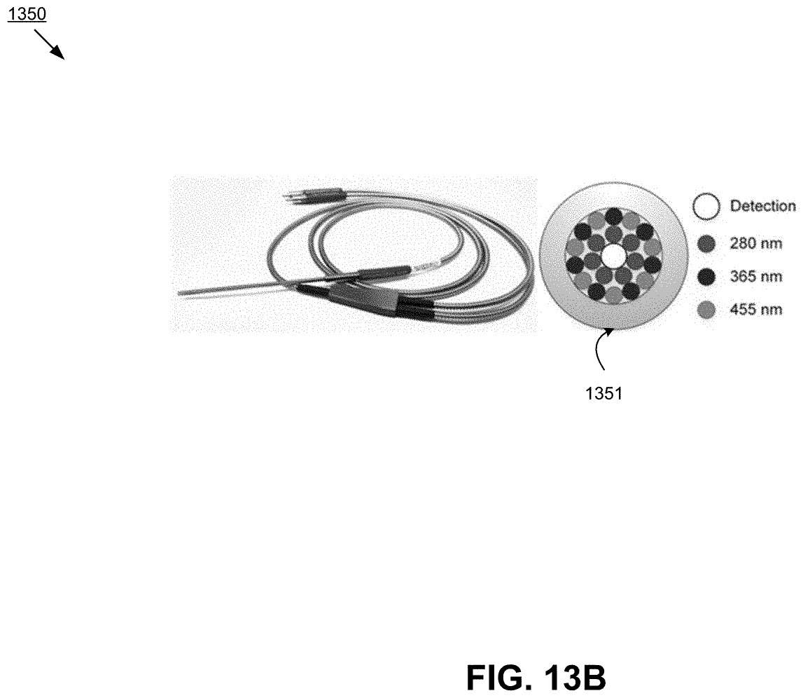

FIG. 13A illustrates an exemplary layout of sensors contained in a sensor pod 1213 located on one side of the gantry head 1201. For example, as illustrated in FIG. 13A, there are three sensors 1301, 1303, and 1305, which can be a pH sensor, DO sensor, and sensor for spectrometer measurement. The sensor pod 1213 can be located on a side of the gantry head 1201 proximate to the sensor probe unit 1211.

The pH sensor can be a UV-VIS spectrometer, and can be used to measure the pH of the growth media. In one example, the sensor probe can be a USB-powered spectrometer capable of measuring wavelengths in the range of 200-850 nm. A pH sensor generally has the most optical activity in the 600-620 nm range for accurate pH reading. To measure the pH value, a light source for illuminating the media sample can be provided. The light source can be a fiber optic light source that can be used to illuminate the pH sticker or dot disposed on one side of the lid for media contact, as described earlier with reference to FIG. 5. The light source can be guided towards the sticker or dot through the probe channel 505 leading to the optical window for illuminating the pH sticker or dot.

To measure the pH value for the media inside a bioreactor unit, the gantry head 1201 can dip the probe unit 1211 (or a pH probe of the probe unit) (including the pH sensor's light source) into the corresponding probe channel in the lid. The light source can then illuminate the pH sticker or dot immersed in the media. The light emitted from the sticker or dot can then be collected by the light probe placed in the probe channel for pH measurement.

The DO sensor can measure the dissolved oxygen of the media by using light to probe the interaction between the DO sticker or dot and the media. For example, a LED light source can illuminate the DO sticker or dot immersed into the growth media. The light emitted from the sticker or dot can be then collected by the DO sensor probe. The DO value may be measured in a similar manner as the pH value. For example, to measure the DO value for the media inside a bioreactor unit, the gantry head 1201 can dip the probe unit 1211 (or a DO probe of the probe unit) (including the DO sensor's light source) into the corresponding probe channel in the lid. The light source can then illuminate the DO sticker or dot immersed in the media. The light emitted from the sticker or dot can then be collected by the light probe placed in the probe channel for DO measurement. In some embodiments, these DO and pH measurements can be collected within a short time span. For example, one measurement can be done right after the other.

The spectrometer can be used to develop a reward function for use in learning and steering the evolutionary dynamics of the biological organisms in the bioreactor wells. The spectrometer can be mounted on the gantry, and can be equipped with UV LEDs to illuminate the sample. A separate optical head can channel the illumination light and carry back the reflected signal to the spectrometer sensor with a portion (e.g., 10%) of the returning signal splitting off to a power meter. An example fiber optical head 1350 for the spectrometer is illustrated in FIG. 13B. As illustrated by the circle 1351 in the figure, the detection and three excitation fiber bundles can be terminated at one sensing probe. The three excitation fiber bundles can have the wavelengths of 280 nm, 365 nm, and 455 nm. The LEDs for emitting the lights with the three wavelengths can be USB powered. In some embodiments, to drive the three LEDs for the spectrometer, a customized or readily available LED driver and LED connection hub can be used. The LED driver can be a 4 channel LED driver, which can supply a current of up to 1 A for each channel independently to a high-power LED.

The measurement of spectrometer wavelengths can be performed in a similar manner and at about the same time as the measurements of the pH and DO values. For example, the gantry head 1201 can dip the spectrometer's optical head into the corresponding probe channel in the lid. The light source can then illuminate the contents of the well. The return light can then be collected by the spectrometer's optical head.

In some embodiments, the three light probes can be combined into one or two probes, to further simplify the configuration of the disclosed bioreactor system.

In some embodiments, a scheduling system can be used to monitor and record sensor readings from in-situ sensors (e.g., for OD and temperature) and/or from the gantry sensors (e.g., for DO, pH, and multi-wavelength spectrometer) in a time coordinated manner based on the start of an epoch. Additionally, in some embodiments, this scheduling system can coordinate with the dispensing system to schedule sensor readings to be taken at times when a dispense operation is not in progress.

In some embodiments, the timing or schedule for collecting DO, pH, and spectrometer measurements can be predefined. For example, for each bioreactor, DO, pH, and multiwavelength spectroscopic measurements can be obtained according to a predefined schedule (e.g., once an hour, twice an hour, or every 45 minutes, etc.). In some embodiments, DO, pH, temperature, OD, and multiwavelength spectroscopic measurements are collected from each bioreactor at least once during each generation of growth. In some embodiments, the DO, pH, and multiwavelength spectroscopic measurements can be obtained at about the same time when the OD and temperature measurements for a bioreactor unit are obtained. For example, for each bioreactor, OD, DO, pH, temperature, and multiwavelength spectroscopic measurements can be obtained within 60 seconds of each other, so that the information can be comparable and temporally correlated during the data analysis. In some embodiments, the measurements collected by the gantry sensors from a bioreactor well are collected serially (rather than in parallel) to avoid optical interference between the sensors.

From the above descriptions, it can be seen that the gantry electronics can include a motor control board operable to selectively position the mobile gantry head at a plurality of positions corresponding to each of the bioreactor units within the set of bioreactor clusters corresponding to the gantry system, a seed dispensing system for seeding each of the bioreactor units, and a plurality of sensor systems and their associated support components. In some embodiments, to reduce the mass of the gantry head 1201, some of the sensor support electronics are not mounted on the head, but rather to the side of the gantry. In particular, the 4 channel LED driver and the associated components (e.g., power meter) can be fixed to the side of the gantry, to allow a more efficient movement of the gantry head 1201 along the rails. In some embodiments, the gantry system can also include an HMI interface for indicating the status of sensors. In some embodiments, the gantry system can further include a gantry controller, as previously described. The gantry controller is operable to use the above described gantry-level sensing devices to obtain measurements of the environments within the bioreactor units.

### 2.4 Stacks of Groups (Stations)

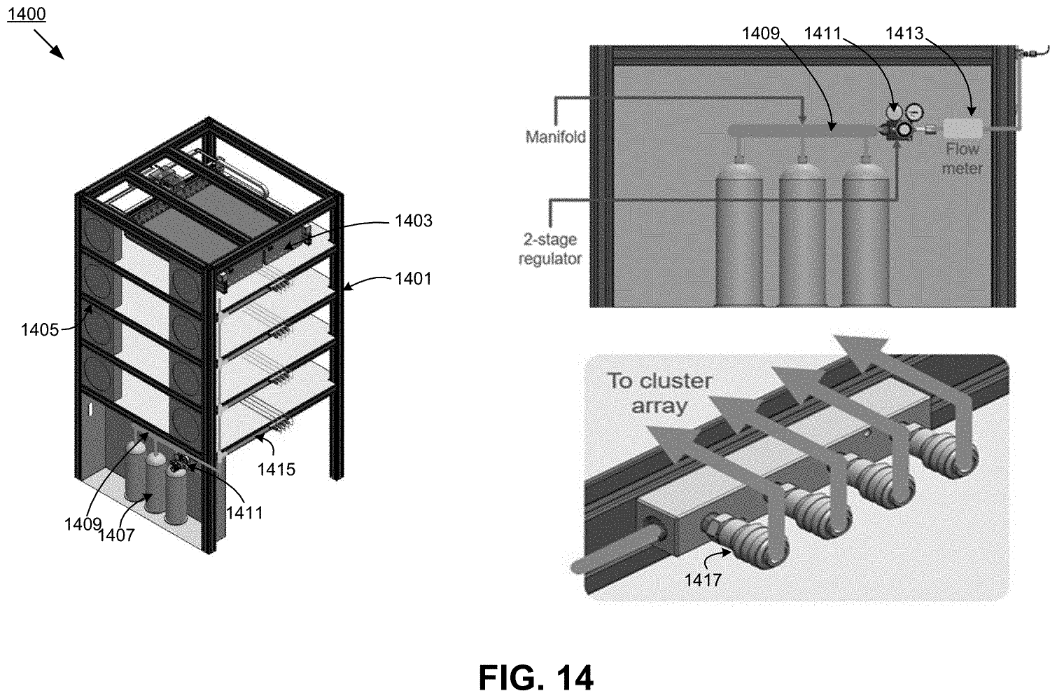

In some embodiments, multiple gantry systems (each gantry servicing a group of clusters of bioreactors) can be stacked to form a bioreactor station 1400, as shown in the left part of FIG. 14. By stacking the gantry systems and bioreactor groups, the footprint of the disclosed bioreactor system 100 can be reduced. For example, the vertical shelving unit 1401 in FIG. 14 can support up to 4 gantry systems 1403 and the corresponding bioreactor groups. Additionally, each gantry system 1403 can utilize a telescoping rail system 1405 that allows the gantry system to be protracted from the shelving unit and serviced with ease.

In some embodiments, oxygen routing can be managed at the bioreactor station level. As shown in the left part of FIG. 14, the bottom portion of the bioreactor station can be dedicated to storing components that service multiple bioreactor groups. In one example, multiple oxygen tanks 1407 can be placed in the bottom portion.

In some embodiments, several oxygen tanks 1407 can be connected to a station oxygen manifold 1409. The output pressure of the manifold can be managed by a 2-stage regulator 1411, as also shown in the top right part of FIG. 14. The inclusion of this 2-stage regulator 1411 allows for constant pressure to be maintained with no readjustment of the regulator. A flow meter 1413 downstream from the regulator 1411 allows the system to calculate the remaining oxygen in the tanks, so they can be swapped out accordingly.

In some embodiments, following the flow meter 1413, oxygen travels up the bioreactor station to the manifolds 1415 located at each level. Each manifold 1415 can service one group of bioreactors. In some embodiments, each manifold 1415 has a number of outlets 1417 that corresponds to the number of bioreactor clusters included in the bioreactor group at each level. For example, there are four outlets 1407 in the bottom right part of FIG. 14. Each of the outlets 1417 provides oxygen to one of the clusters of bioreactors included in the group of bioreactors.

In some embodiments, the disclosed bioreactor system 100 can scale up the number of bioreactor stations so that the total number of bioreactor units can be scaled up to a desired number of bioreactor units for an evolution dynamics study. For example, if the illustrated station holds 1024 (64×4×4) bioreactor units in the station, to meet a requirement of at least 10,000 bioreactor units, 10 of such bioreactor stations can be grouped together, which provides 10,240 bioreactor units for an evolution dynamics study.

### 3. BIOREACTOR SYSTEM

In some embodiments, different stations are grouped together physically (e.g., in a same room or same location), and/or systematically. For example, these different stations can be communicationally coupled to each other. In addition, all these stations can be managed by a single controlling system, which can control all the bioreactor units included in these stations, as well as collecting data from these different stations, so that techniques for learning and steering evolutionary dynamics can be applied to the biological organisms in all the bioreactor wells of the disclosed large scale bioreactor system.

For example, to obtain sufficient data for learning and steering evolutionary dynamics, the controlling system can determine the selective pressures to be applied, control the application of specific selective pressures to specific bioreactor units at each epoch, and evaluate each population's evolution at each epoch of organism growth, and then determine the selection pressures to be applied at the next epoch, to further learn and steer the evolutionary dynamics of the organisms.

The high level control of the disclosed bioreactor system can be implemented using main control algorithms that run on a local server or on a remote server (e.g., Amazon Web Service (AWS)). Such a local or remote server can be also referred to as the master controller for the disclosed bioreactor system.

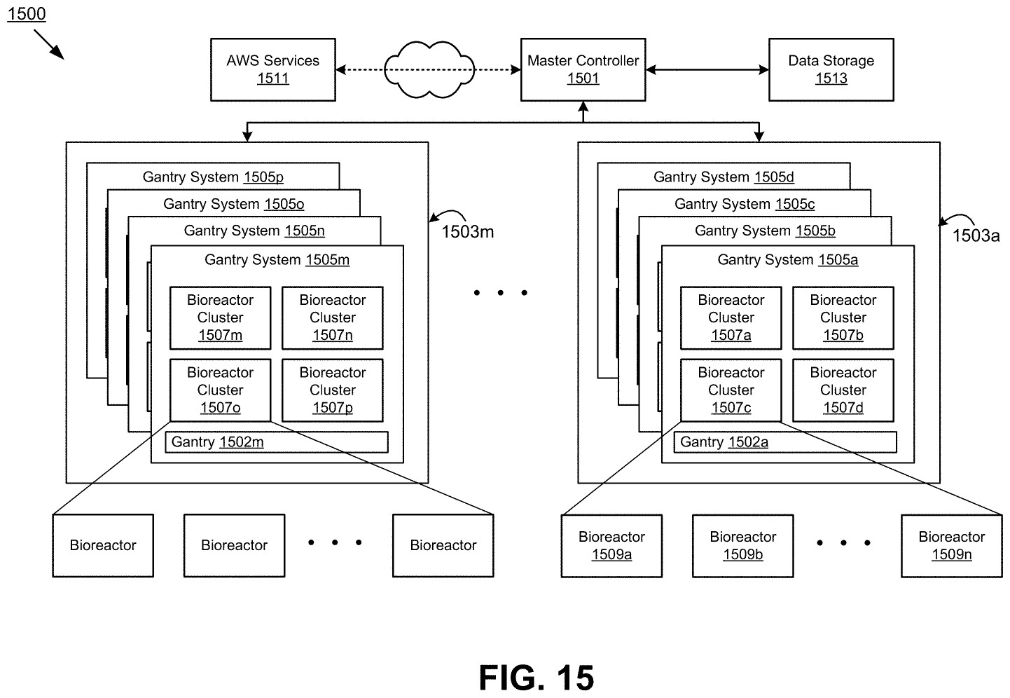

FIG. 15 illustrates a high level architecture of a bioreactor system 1500 containing a master controller, according to some embodiments. As illustrated, the bioreactor system 1500 includes a master controller 1501 that manages a number of bioreactor stations 1503a . . . 1503m (together or individually referred to as bioreactor station 1503). Each bioreactor station includes a number of gantry systems (or “bioreactor groups”), such as gantry systems 1505a-1505d or 1505m-1505p (together or individually referred to as gantry system 1505). Each gantry system 1505 includes a number of bioreactor clusters, such as bioreactor clusters 1507a-1507b or 1507m-1507p (together or individually referred to as bioreactor cluster 1507), and a gantry 1502. Each bioreactor cluster further includes a number of bioreactors, such as 1509a-1509n (together or individually referred to as bioreactor 1509).

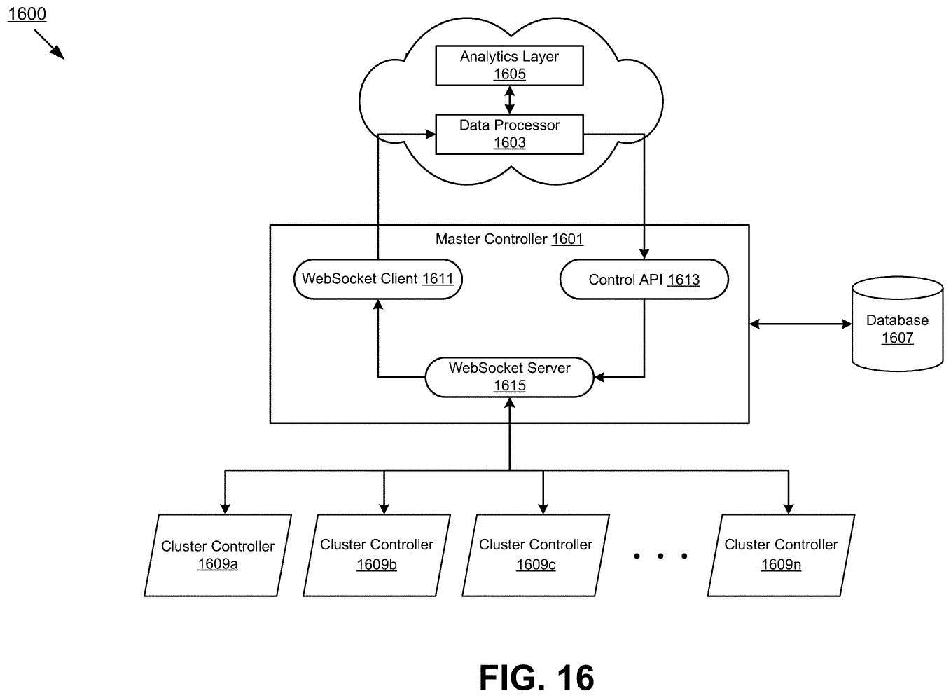

The master controller 1501 can aggregate the data from all bioreactors 1509, and store the aggregated locally in the data store 1513, in case there are connectivity problems with the cloud. In some embodiments, the data can also be sent to the cloud for backup storage. In some embodiments, the master controller 1501 also listens for commands and relays the instructions to the bioreactor clusters 1507. For example, the master controller 1501 can send instructions to the cluster controllers to instruct the gantry systems 1505 to collect OD signals to assess organism growth. The master controller 1501 is described in greater detail below with reference to FIG. 16.

FIG. 16 illustrates an exemplary hardware/software architecture 1600 of the bioreactor system, according to some embodiments. As illustrated, the exemplary architecture 1600 can be divided into three layers: master controller 1601, data processor 1603, and analytics layer 1605. In FIG. 16, the master controller 1601 comprises, is a component of, or is executed by a computer local to the bioreactor clusters, while the data processor 1603 and the analytics layer 1605 run in the cloud. In some embodiments, the data processor 1603 and/or the analytical layer 1605 can comprise, be components of, or be executed by a computer local to the bioreactor clusters as well, for example, on a same computer that runs the master controller 1601. Additionally or alternatively, the master controller 1601 can run on a remote server, such as AWS as described earlier.

In some embodiments, the master controller 1601 also connects to the cluster controllers 1609a, 1609b, 1609c, . . . , 1609n (together or individually referred to as cluster controller 1609) associated with the bioreactor clusters included in various bioreactor stations. Each of the cluster controllers 1609 may be, for example, a cluster control board as described above. The master controller 1601 can receive data from the cluster controllers 1609 through a WebSocket server 1615, and store the data in a database 1607 coupled to the master controller 1601. In some embodiments, the master controller 1601 also streams the data to the data processor's WebSocket server (not shown) in the cloud through a WebSocket client 1611 interface. The master controller 1601 can receive commands from the data processor 1603 via a control API 1613 (e.g., representational state transfer (REST) API). The control API 1613 can forward the commands to the appropriate cluster controller(s) 1609 through the WebSocket server 1615.

In some embodiments, the master controller 1601 comprises a JavaScript web server running in Node.JS installed on a computer. It can connect to a PostgreSQL database server running on the same computer or a separate, dedicated server computer.

In some embodiments, the analytics layer 1605 further includes an analytics algorithm that can process the data received by the data processor 1603. In some embodiments, the analytics layer 1605 can pass commands to the data processor 1603. In some embodiments, the master controller 1601 does not interact directly with the analytics layer 1605.

In some embodiments, the cluster controllers 1609 automatically connect to the WebSocket server 1615 on the master controller 1601. Accordingly, the master controller 1601 can construct a logical map of the system topology made up of the cluster controllers 1609 and their associated bioreactors.

In some embodiments, the cluster controller WebSocket allows the master controller 1601 to receive and access all the real time data being generated by the bioreactors and the bioreactor clusters 1609. This information can be stored in the database 1607 and sent to the data processor 1603. In some embodiments, the cluster controller WebSocket server 1615 also allows the master controller 1601 to send commands to the bioreactor clusters.

In some embodiments, the control API 1613 exposed by the master controller 1601 allows for any settings to be changed on the cluster controllers 1609 and bioreactors. In some embodiments, the control API 1613 can be exposed to the data processor 1603, and can also be accessed by a REST client associated with the master controller computer when needed.

The database 1607 can accumulate all the data collected from a “run” of the bioreactor system (e.g., a period of operation of the bioreactor system comprising a plurality of epochs in which the evolutionary dynamics of a population of organisms are learned and steered). For example, the database 1607 can store all the data collected from sensors, status updates from the cluster controllers 1609, and a transaction record of all actions taken by the bioreactor system 100.

In some embodiments, the architecture 1600 of the disclosed biosystem 100 can further include diagnostic software, which can be used by operators and/or third parties to service and troubleshoot problems in the bioreactor system. In some embodiments, the diagnostic software can be run on all computational units on the bioreactor system 100.

For example, to facilitate debugging, an extensive logging system can be configured to gather data from all the processing elements and store it to the master controller 1601. This information can then be accessed remotely for diagnosing errors or other issues in the system.

The information being logged can include all the diagnostic sensors information, plus other data that can be valuable for diagnosing problems. An HMI type software package can be run on the master controller to allow users or third parties to debug errors or issues within the system.

In some embodiments, during startup, a power-on self-test (POST) can be used to identify potential problems inside the system. During this time, it is often possible to conduct tests that would be difficult to conduct during the normal operation of the device. The focus of the diagnostic software can be on gathering and storing diagnostic and status information about the system 100.

Accordingly, in some embodiments, the bioreactor system 100 can further include certain diagnostic sensors for collecting diagnostic information. Diagnostic sensors can be used to determine if there are problems in the system, and can further assist with the rapid identification and assessment of faults if there are problems. In addition, the diagnostic sensors can also help with serviceability between runs. For example, the liquid level can be sensed via capacitive sensing at the sidewall of each bioreactor. The types of diagnostic sensors envisioned in the system include, but are not limited to, connectivity, current, leak, light, temperature, and watchdog.

Connectivity sensors can monitor the system for network failures. By using the master controller and the HMI on the bioreactor clusters, it may be possible to quickly track down connection failures between the master controller and the bioreactors clusters. It may also allow for the quick location of failures in the communication between the bioreactor cluster PCB and the bioreactor boards. Current sensors are good at detecting failures in actuators and sensors. By monitoring the current inside the various components in the system, it is possible to detect potential failures quickly.

Leak sensors can facilitate the detection of connection failures in plumbing, possible cracks or sealing problems in the manifolds. Leak sensors can be positioned in a location that can sense liquid pooling below the bioreactor clusters. If a leak is observed on the system, an emergency stop switch (E-stop) can be used to interrupt power to the pumps. Also, a liquid level sensor's environment sensor input can be monitored to determine if the liquid in a bioreactor is in an overflow state. Likewise, a liquid level sensor can be used to detect if the liquid level in a bioreactor is below a threshold level (e.g., below the bottom of the level sensor). In some embodiments, software diagnostic features can include identifying leaks by correlating changes in a bioreactor's liquid level relative to a rate of liquid flow. If the liquid level in the bioreactor is lower than expected based on liquid flow rate, a slow leak may be indicated.

A gantry power meter can also be used to monitor excitation sources. A failure in an excitation LED can lead to erroneous operation of the unit. Temperature sensors can be used to monitor boards or components for possible malfunction. A board that is getting too hot could indicate a component failure. A failure in the heating or cooling system can also be seen with unusual temperature offsets. Watchdogs can monitor the behavior of the computation units in the system. If a microcontroller fails to feed the watchdog, the watchdog can reset the unit. This approach can prevent a software or hardware error from causing a unit to stop responding.

In some embodiments, two redundant sensor technologies can be used in the disclosed bioreactor system 100. That is, for each type of measurement, two similar sensors can be used, which also provide information for diagnostic purposes.

In some embodiments, there is an additional system-level cleaning process configured for the disclosed bioreactor system 100 to ensure that the system can be ready for the next run. In some embodiments, cleaning of the bioreactor system 100 can be performed with a wash in-situ leveraging the feed, aux, O₂, and waste channels as well as the stirring and heating mechanisms. For example, instead of dispensing growth media and aux, the cleaning solution and the distilled water can be dispensed into the wells during a clean process. For another example, instead of dispensing oxygen into a well, steam can be dispensed into each well during the cleaning process.

In some embodiments, the bioreactor lids can be hand washed. Alternatively, disposing of and replacing the entire lids is an option. Due to the disposable nature of the pH and DO sensor “dots,” removing, disposing and replacing of the light pipes is an option. The covers for the optical density, optical temperature, and optical level sensor can be disposable as well. The use of removable and/or disposable covers may facilitate reuse of sensors (e.g., the optical density sensor) from run to run without risk of cross contamination.

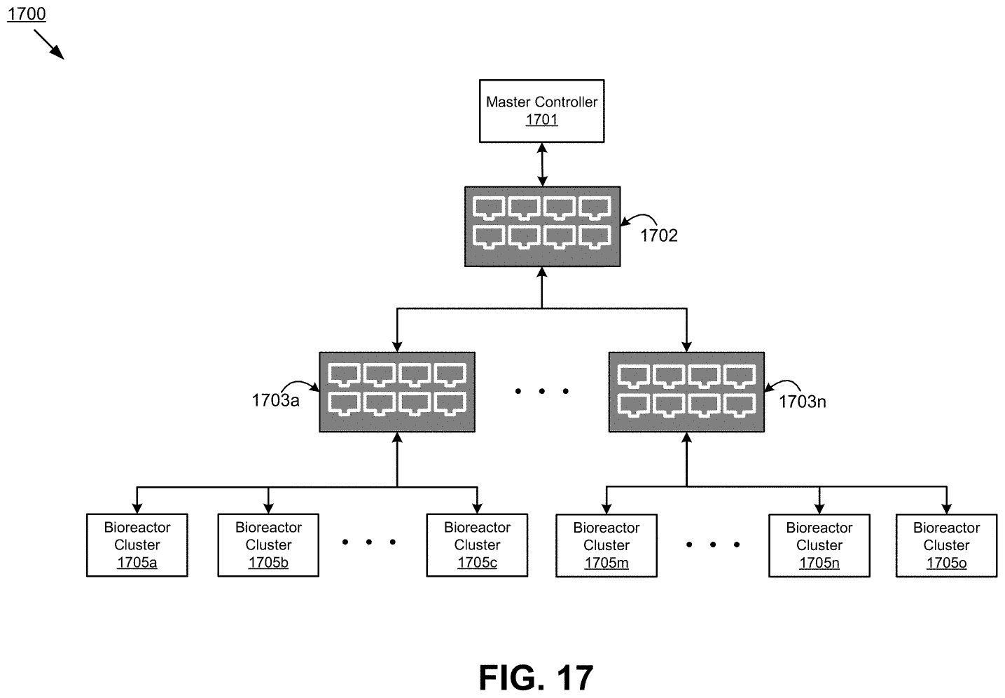

Referring to FIG. 17, a communication network 1700 between the master controller and bioreactor clusters is further presented, according to some embodiments. The estimated bandwidth from a master controller 1701 to all the bioreactor clusters 1705a . . . 1705o (together or individually referred to as bioreactor cluster 1705) can be less than 300 KB/s. A 1 Gb/s second network can operate at less than 1% of the total available bandwidth to support this type of data rate. As such, the platform can be configured to minimize congestion on the network 1700 and give the network 1700 an almost real-time performance. In an example, even a 1-second delay may not be a major concern with regard to network delay.

The master controller 1701 can be connected to the individual bioreactor cluster 1705 via an Ethernet networking interface. In an embodiment, a 2 layer network system 1700 can be used. In an example, assuming 64 bioreactors per bioreactor cluster, and 16-port switches, the 2-layer communication network can cover over 14,000 bioreactors.

This type of network offers the following advantages: (1) it is inexpensive, electrically isolated, fast, robust, and expandable; (2) it has high bandwidth; (3) messages can travel tens of meters over the network; (4) it can support several different protocols; and (5) different parts of the network can run at different speeds.

In some embodiments, the bioreactor system can operate properly without real time delivery of messages. Referring to FIG. 17, the master controller 1701 can interface to the top switch 1702 with a bandwidth of at least 1 Gb/s. Likewise, the communication from the second layer switches 1703a . . . 1703n to the top switch can be at least 1 Gb/s. In some embodiments, the speed from each bioreactor cluster to the 2nd layer switch can be at least 100 Mb/s.

In some embodiments, the disclosed bioreactor system 100 further includes reservoirs for the dispensable materials (e.g., O₂, aux, and media), for waste, and for the ancestral population used to seed the bioreactors. The ancestral population can be stored or be growing in a sealed sterile vessel or in a bioreactor (e.g., a conventional bioreactor) that holds a constant stable ancestor population that is used to seed all the bioreactors in the disclosed bioreactor system. In some embodiments, the sealed sterile vessel or an ancestral bioreactor can be connected to the seeding syringes included in the gantry heads by channels and valves.

In some embodiments, multiple ancestral reservoirs can be used to seed the bioreactors in a run of the bioreactor system. For example, if an operator wants to simultaneously run an analysis using different seeding populations (e.g., different organisms), there can be multiple ancestral reservoirs for the different organisms.

The materials including media and aux for organism growth can be stored in a sealed sterile tank. The vessel and tanks can be large enough to last an entire run, so that the refilling can be performed after a run completes, rather than during each run. To prevent a run from abortion, in the event that media or buffer runs low, the media or buffer can be added to the tanks from another sealed sterile tank through sterile pumps and connection tubes, or through other different sterile approaches. In some embodiments, waste can be also stored in a tank, which is generally large enough to hold a full run worth of waste, so that disposal can be performed until after a run completes.

### 4. SOME EXAMPLES OF A COMPUTER SYSTEM

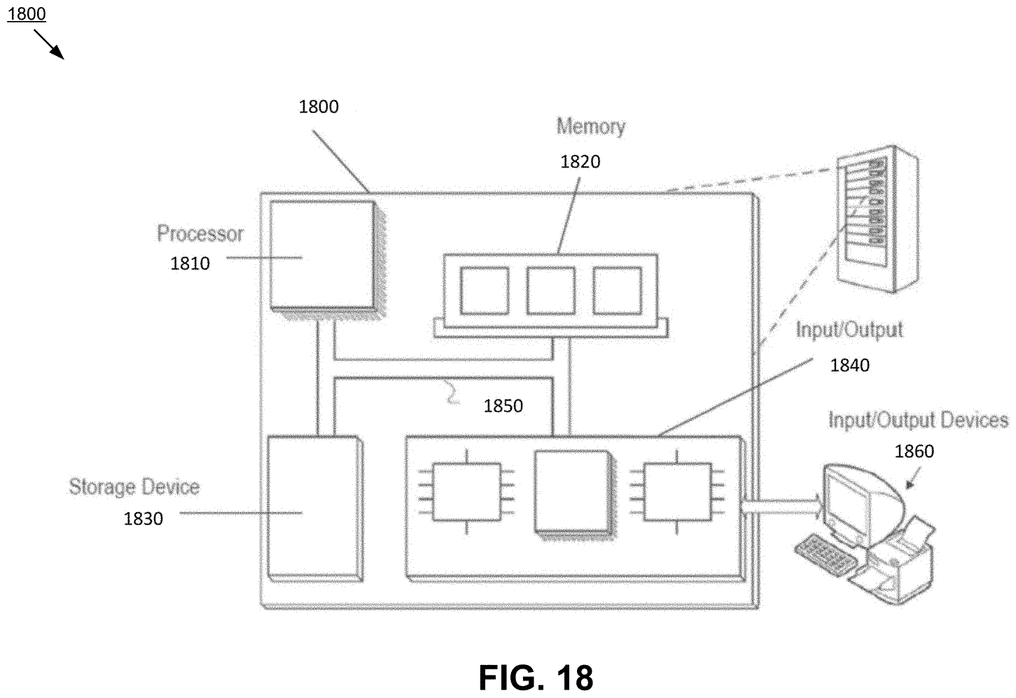

Referring now to FIG. 18, a block diagram of an example computer system 1800 that may be used in implementing the technology is provided. General-purpose computers, network appliances, mobile devices, or other electronic systems may also include at least portions of the system 1800. The system 1800 includes a processor 1810, a memory 1820, a storage device 1830, and an input/output device 1840. Each of the components 1810, 1820, 1830, and 1840 may be interconnected, for example, using a system bus 1850. The processor 1810 is capable of processing instructions for execution within the system 1800. In some implementations, the processor 1810 is a single-threaded processor. In some implementations, the processor 1810 is a multi-threaded processor. The processor 1810 is capable of processing instructions stored in the memory 1820 or on storage device 1830.

The memory 1820 stores information within the system 1800. In some implementations, the memory 1820 is a non-transitory computer-readable medium. In some implementations, the memory 1820 is a volatile memory unit. In some implementations, the memory 1820 is a non-volatile memory unit.

The storage device 1830 is capable of providing mass storage for the system 1800. In some implementations, the storage device 1830 is a non-transitory computer-readable medium.

In various different implementations, the storage device 1830 may include, for example, a hard disk device, an optical disk device, a solid-date drive, a flash drive, or some other large capacity storage devices. For example, the storage device may store long-term data (e.g., database data, file system data, etc.). The input/output device 1840 provides input/output operations for the system 1800. In some implementations, the input/output device 1840 may include one or more of network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices 1860. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 1830 may be implemented in a distributed way over a network, such as a server farm or a set of widely distributed servers, or may be implemented in a single computing device.

Although an example processing system has been described in FIG. 18, embodiments of the subject matter, functional operations and processes described in this specification can be implemented in other types of digital electronic circuitry, in tangibly-embodied computer software or firmware, in computer hardware, including the structures disclosed in this specification and their structural equivalents, or combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, an engine, a software module, a script, or code) can be written in any form of programming language, including compiled or interpreted languages, or declarative or procedural languages, and it can be deployed in any form, including as a standalone program or as a module, component, subroutine, or other units suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit can receive instructions and data from a read-only memory or a random access memory or both. A computer generally includes a central processing unit for performing or executing instructions and one or more memory devices for storing instructions and data. Generally, a computer can also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### 5. DESCRIPTION OF SOME EMBODIMENTS

According to an aspect of the present disclosure, a bioreactor system including one or more bioreactor groups is provided. Each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. The gantry includes one or more sensors and a movable gantry head with one or more sensor probes configured to selectively sense properties of contents of respective bioreactor units included in the plurality of bioreactor units.

In some embodiments, each bioreactor unit has a respective stirring mechanism, temperature control subsystem, temperature sensor, liquid level sensor, and optical density sensor.

In some embodiments, the plurality of bioreactor units included in each of the bioreactor clusters are organized in a number of rows, where each row has a same number of bioreactor units.

In some embodiments, the bioreactor system further includes a master controller board configured to control the bioreactor system, a plurality of cluster controller boards configured to control, respectively, the plurality of bioreactor clusters, and a plurality of bioreactor boards configured to control, respectively, the rows of bioreactor units.

In some embodiments, the master controller board communicates with the cluster controller board associated with the plurality of bioreactor clusters. Each cluster controller board associated with each bioreactor cluster in the plurality of bioreactor clusters communicates with the bioreactor boards associated with the rows of bioreactor units in the respective bioreactor cluster.

In some embodiments, the one or more manifolds included in each bioreactor cluster include a respective top manifold and a respective plurality of bottom manifolds in each bioreactor cluster.

In some embodiments, in each bioreactor cluster, each of the plurality of bottom manifolds corresponds to a row of bioreactor units in the respective bioreactor cluster.

In some embodiments, in each bioreactor cluster, the one or more dispensing valves of the bioreactor units in the respective bioreactor cluster are disposed within the top manifold.

In some embodiments, in each bioreactor cluster, the waste valves of the bioreactor units in each row of the respective bioreactor cluster are disposed within a corresponding bottom manifold.

In some embodiments, in each bioreactor cluster, the top manifold further includes one or more fluid channels configured to provide the one or more materials to the bioreactor units in the respective bioreactor cluster.

In some embodiments, in each bioreactor cluster, the one or more fluid channels include a fluid channel configured to provide growth media to the bioreactor units in the respective bioreactor cluster, a fluid channel configured to provide an auxiliary solution to adjust pH of growth media in the wells of the bioreactor units in the respective bioreactor cluster, and/or a fluid channel configured to provide oxygen to the bioreactor units in the respective bioreactor cluster.

In some embodiments, in each bioreactor cluster, each bottom manifold further includes a fluid channel configured to remove the waste drained from the bioreactor units in the respective row of the bioreactor cluster corresponding to the respective bottom manifold.

In some embodiments, the one or more sensors included in the gantry include a pH sensor, a dissolved oxygen sensor, and/or a spectrometer.

In some embodiments, the lid for each bioreactor unit includes one or more probe channels for inserting one or more of the pH sensor, the dissolved oxygen sensor, or the spectrometer sensor into the lid from the gantry.

In some embodiments, the lid for each bioreactor unit further includes one or more stickers configured to facilitate detection of one or more of a pH value or a dissolved oxygen concentration inside the well of the respective bioreactor unit.

In some embodiments, each gantry head is operable to move to a position corresponding to each bioreactor unit included in the respective group of bioreactor clusters corresponding to the gantry head.

In some embodiments, the one or more bioreactor groups include a plurality of bioreactor groups stacked vertically to form a bioreactor station.

In some embodiments, the bioreactor system includes 10 bioreactor stations, where each bioreactor station includes four bioreactor groups, each bioreactor group includes four bioreactor clusters, and each bioreactor cluster includes 64 bioreactor units.

In some embodiments, the 64 bioreactor units in each bioreactor cluster are organized in an 8×8 array.

According to another aspect of the present disclosure, a bioreactor system including one or more bioreactor groups is provided. Each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. Each bioreactor has an independent temperature control subsystem.

In some embodiments, each independent temperature control subsystem includes one or more thermoelectric coolers (TECs) configured to control temperature of an associated bioreactor unit.

In some embodiments, each independent temperature control subsystem includes one or more insulation materials disposed between adjacent bioreactor units.

In some embodiments, each independent temperature control subsystem includes an air gap disposed between adjacent bioreactor units

In some embodiments, the one or more manifolds are made of thermal resistant material.

In some embodiments, the one or more TECs control the temperature for the associated bioreactor unit by heating and/or cooling the associated bioreactor unit.

In some embodiments, each bioreactor unit further includes a temperature sensor configured to measure a temperature of the respective bioreactor unit.

In some embodiments, the one or more TECs heat and/or cool the associated bioreactor unit based on the measured temperate for the associated bioreactor unit.

In some embodiments, each of the temperature sensors is a non-contact infrared-based sensor.

In some embodiments, each of the temperature sensors has a measurement range between 0-50 degrees Celsius.

In some embodiments, each of the temperature sensors is disposed on one side of the associated bioreactor unit outside the well of the associated bioreactor unit.

In some embodiments, each of the temperature sensors is mounted on a printed circuit board disposed on one side of the associated bioreactor unit.

In some embodiments, each of the temperature sensors is further configured to monitor temperatures of one or more bioreactor components other than the associated bioreactor unit.

In some embodiments, each of the temperature sensors is configured to generate an alert signal if a measured temperature is outside a specified range.

In some embodiments, each independent temperature control subsystem further includes a passive heat distributor configured to distribute heat generated by the one or more TECs.

In some embodiments, each passive hear distributor includes one or more of a cold finger or a heat sink.

In some embodiments, each cold finger or heat sink has a shape that conforms to an outer surface of a well of the associated bioreactor unit.

In some embodiments, the bioreactor system further includes one or more fans configured to circulate ambient air around the bioreactor system.

In some embodiments, adjacent bioreactor units in a bioreactor cluster are separated by at least a specified distance.

In some embodiments, the specified distance is determined based on a simulation result from a thermal analysis of the bioreactor cluster.

According to another aspect of the present disclosure, a bioreactor system including one or more bioreactor groups is provided. Each group has a plurality of bioreactor clusters and a gantry shared by the plurality of bioreactor clusters. Each bioreactor cluster includes a plurality of bioreactor units organized in rows. Each bioreactor unit has a well, a lid covering the well, a waste valve configured to control draining of waste out of the well, and one or more dispensing valves configured to control dispensing one or more materials into the well. Each bioreactor cluster further includes a top manifold shared by the plurality of bioreactor units included in the respective bioreactor cluster, and a plurality of bottom manifolds, each bottom manifold corresponding to a row of bioreactor units within the respective bioreactor cluster.

In some embodiments, the one or more dispensing valves for controlling the dispensing of the materials are disposed within the top manifold.

In some embodiments, the waste valve for removing the waste out of each well is disposed within one of the plurality of bottom manifolds.

In some embodiments, each bioreactor unit further includes a plurality of unit fluid channels formed in the top manifold and in fluidic communication with the well.

In some embodiments, the plurality of unit fluid channels includes a unit fluid channel for dispensing growth media into the well, a unit fluid channel for dispensing an auxiliary solution to adjust pH of growth media inside a well, and a unit fluid channel for dispensing oxygen into growth media inside a well.

In some embodiments, each unit fluid channel has a corresponding dispensing valve for controlling a flow rate of dispensing a respective material into the well.

In some embodiments, the top manifold further includes a plurality of row fluid channels for each row of bioreactor units, where each row fluid channel is configured to dispense a respective material into wells within the row of bioreactor units.

In some embodiments, each row fluid channel is in fluidic communication with respective unit fluid channels included in that row.

In some embodiments, the top manifold further includes a plurality of cluster fluid channels. Each cluster fluid channel is configured to dispense a respective material into respective row fluid channels for dispensing the respective material into rows of bioreactor units.

In some embodiments, the plurality of cluster fluid channels include an auxiliary cluster fluid channel, a growth media cluster fluid channel, and an oxygen cluster fluid channel.

In some embodiments, each of the cluster fluid channels is in fluidic communication with a respective reservoir, for storing a respective material, through a respective reservoir manifold.

In some embodiments, a reservoir manifold includes a flow sensor for detecting a flow rate of a respective material passing through the reservoir manifold.

In some embodiments, a reservoir manifold includes a pump for pumping a respective material from a respective reservoir to a respective cluster fluid channel.

In some embodiments, each bioreactor cluster further includes a cluster controller board.

In some embodiments, each bioreactor cluster further includes a plurality of bioreactor boards, each bioreactor board corresponding to a row of bioreactor units.

In some embodiments, the cluster controller board is configured to control dispensing of a target material to a target bioreactor unit included in the bioreactor cluster.

In some embodiments, the bioreactor board controls the dispensing of the target material to the target bioreactor unit by closing all valves except a value corresponding to the target material and the target bioreactor unit, and turning on a respective pump for pumping the target material from a respective reservoir.

In some embodiments, each bioreactor unit further includes a unit fluid channel formed in a respective bottom manifold and in fluidic communication with the well.

In some embodiments, each bottom manifold further includes a row fluid channel in fluidic communication with unit fluid channels included in that row.

According to another aspect of the present disclosure, a bioreactor unit including a well and a lid covering the well is provided. The lid further includes a pierceable lid septum configured to permit injection of a seeding solution into the well. The bioreactor unit further includes one or more inlets configured to dispense materials into the well. Each inlet has a corresponding valve configured to control dispensing of material. The bioreactor unit further includes an outlet configured to drain waste from the well. The outlet has a corresponding valve configured to control draining of waste. The bioreactor unit further includes a fluid level sensor configured to sense a volume of contents of the well.

According to another aspect of the present disclosure, a bioreactor system is provided. The bioreactor system includes a plurality of bioreactor units including a respective plurality of wells, a bioreactor controller board including a plurality of temperature sensors and optical density sensors corresponding, respectively, to the plurality of bioreactor units. Each temperature sensor is configured to sense a temperature of a well of the respective bioreactor unit and each optical density sensor is configured to sense an optical density of contents of the respective bioreactor unit. The bioreactor system further includes a plurality of lids configured to cover, respectively, the plurality of wells. Each lid includes a plurality of probe channels configured to extend downward from an upper surface of the lid into the respective well. Each of the probe channels is configured to guide an optical probe from the upper surface of the lid to a position proximate to a bottom of the probe channel.

According to another aspect of the present disclosure, a bioreactor system is provided. The bioreactor system includes a plurality of bioreactor clusters, where each of the bioreactor clusters includes a plurality of bioreactor units and a gantry shared by the plurality of bioreactor clusters. The gantry includes a mobile gantry head, and a plurality of sensors. Each sensor includes a respective sensor probe, where the sensor probes are mounted on the mobile gantry head. The gantry is configured to selectively position the gantry head above each bioreactor unit in the plurality of bioreactor clusters, insert the sensor probes into corresponding probe channels in a lid of the respective bioreactor unit, and sense properties of contents of the respective bioreactor unit.

According to another aspect of the present disclosure, a method for forming a bioreactor system is provided. The method includes forming one or more bioreactor groups, where each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. The gantry includes one or more sensors and a movable gantry head with one or more sensor probes configured to selectively sense properties of contents of respective bioreactor units included in the plurality of bioreactor units.

According to another aspect of the present disclosure, a method for forming a bioreactor system is provided. The method includes forming one or more bioreactor groups, where each group has a plurality of bioreactor clusters and a gantry shared by the plurality of bioreactor clusters. Each bioreactor cluster includes one or more manifolds and a plurality of bioreactor units. Each bioreactor unit has a well, a lid covering the well, a waste valve configured to control draining of waste out of the well, and one or more dispensing valves configured to control dispensing of one or more materials into the well. Each bioreactor further includes an independent temperature control subsystem.

According to another aspect of the present disclosure, a method for forming a bioreactor system is provided. The method includes forming one or more bioreactor groups, where each group has a plurality of bioreactor clusters and a gantry shared by the plurality of bioreactor clusters. Each bioreactor cluster includes a plurality of bioreactor units organized in rows. Each bioreactor unit has a well, a lid covering the well, a waste valve configured to control draining of waste out of the well, and one or more dispensing valves configured to control dispensing one or more materials into the well. Each bioreactor cluster further includes a top manifold shared by the plurality of bioreactor units included in the respective bioreactor cluster, and a plurality of bottom manifolds, each bottom manifold corresponding to a row of bioreactor units within the respective bioreactor cluster.

According to another aspect of the present disclosure, a method for forming a bioreactor unit is provided. The method includes forming a well, forming a lid covering the well, where the lid further includes a pierceable lid septum configured to permit injection of a seeding solution into the well; forming one or more inlets for dispensing materials into the well, where each inlet has a corresponding valve configured to control dispensing of material; forming an outlet for draining waste from the well, where the outlet has a corresponding valve configured to control draining of waste; and forming a fluid level sensor for sensing a volume of contents of the well.

According to another aspect of the present disclosure, a method for forming a bioreactor system is provided. The method includes forming a plurality of bioreactor units including a respective plurality of wells, forming a bioreactor controller board, where the bioreactor cluster board includes a plurality of temperature sensors and optical density sensors corresponding, respectively, to the plurality of bioreactor units. Each temperature sensor is configured to sense a temperature of a well of the respective bioreactor unit and each optical density sensor is configured to sense an optical density of contents of the respective bioreactor unit. The method further includes forming a plurality of lids, the plurality of lids being configured to cover, respectively, the plurality of wells, where each lid includes a plurality of probe channels configured to extend downward from an upper surface of the lid into the respective well, and each of the probe channels is configured to guide an optical probe from the upper surface of the lid to a position proximate to a bottom of the probe channel.

According to another aspect of the present disclosure, a method for forming a bioreactor system is provided. The method includes forming a plurality of bioreactor clusters, where each of the bioreactor clusters includes a plurality of bioreactor units, and a gantry shared by the plurality of bioreactor clusters. The gantry includes a mobile gantry head and a plurality of sensors, where each sensor includes a respective sensor probe. The sensor probes are mounted on the mobile gantry head. The gantry is configured to selectively position the gantry head above each bioreactor unit in the plurality of bioreactor clusters, insert the sensor probes into corresponding probe channels in a lid of the respective bioreactor unit, and sense properties of contents of the respective bioreactor unit.

According to another aspect of the present disclosure, a method for operating a bioreactor system is provided. The bioreactor system includes one or more bioreactor groups, where each group has a plurality of bioreactor clusters and a gantry shared by the plurality of bioreactor clusters. Each bioreactor cluster includes a plurality of bioreactor units. Each bioreactor unit includes a well, a lid covering the well and having one or more channel windows, and a first set of sensors disposed on one side of the bioreactor unit. The gantry includes a second set of sensors and a movable gantry head with one or more sensor probes configured to selectively sense properties of contents of respective bioreactor units included in the plurality of bioreactor units. The method includes performing a first measurement of contents inside a target bioreactor unit using one of the first set of sensors disposed adjacent to the target bioreactor unit, and/or performing a second measurement of the contents inside the target bioreactor unit using one of the second set of sensors included in the gantry.

In some embodiments, performing the second measurement of the contents inside the target bioreactor unit using the one of the second set of sensors included in the gantry includes moving the gantry head to a position over the target bioreactor unit, moving the gantry head towards a lid of the target bioreactor unit, to allow a sensor probe associated with the one of the second set of sensors to insert into a corresponding channel window disposed within the lid of the target bioreactor unit, and activating the one of the second set of sensors, to conduct the second measurement of the contents inside the target bioreactor unit.

In some embodiments, the bioreactor system further includes one X-axis guild rail and two Y-axis linear guide rails associated with the gantry, and moving the gantry head to the position over the target bioreactor unit further includes moving the gantry along the two Y-axis linear guide rails, and/or moving the gantry along the X-axis guide rail towards the target bioreactor unit.

In some embodiments, the bioreactor system further includes a cluster controller board associated with the gantry and a motor to drive the gantry head to move, and moving the gantry head to the position over the target bioreactor unit further includes providing motor drive signals to the gantry head by the cluster controller board, to drive the gantry head to move to the position over the bioreactor unit, where the motor drive signals include X, Y, and Z position information regarding where the gantry head will move to.

In some embodiments, the first measurement and second measurement are conducted within a 60 second time window.

In some embodiments, the first set of sensors include a temperature sensor and an optical density sensor, and the second set of sensors include a dissolved oxygen sensor, a pH sensor, and a spectrometer sensor.

According to another aspect of the present disclosure, a method for operating a bioreactor system is provided. The bioreactor system includes one or more bioreactor groups, where each group has a plurality of bioreactor clusters. Each bioreactor cluster includes a plurality of bioreactor units and a plurality of tanks or reservoirs for storing resources and waste materials. Each bioreactor unit includes a well, a lid covering the well, a waste valve configured to control draining of waste out of the well, and one or more dispensing valves configured to control dispensing of one or more materials into the well. The method includes dispensing one of the one or more materials into a well of a target bioreactor unit through controlling the waste valve and the one or more dispensing valves of each bioreactor unit, and/or draining waste from the well of the target bioreactor unit through controlling the waste valve and the one or more dispensing valves of each bioreactor unit.

In some embodiments, each of the plurality of tanks or reservoirs includes a flow sensor and an associated pump, and dispensing the one of the one or more materials into the well of the target bioreactor unit includes closing the waste valve and the one or more dispensing valves associated with each bioreactor unit included in a bioreactor cluster associated with the target bioreactor unit, except one dispensing valve for dispensing the one of the one or more materials to the target bioreactor unit, turning on a corresponding pump for a tank or reservoir storing the one of the one or more materials, monitoring an amount of the materials dispensed to the target bioreactor unit based on information collected from a flow sensor associated with the corresponding pump, and turning off the corresponding pump for the tank or reservoir when a target volume of material is dispensed.

In some embodiments, each of the plurality of tanks or reservoirs includes a flow sensor and an associated pump, and draining the waste from the well of the target bioreactor unit includes closing the waste valve and the one or more dispensing valves associated with each bioreactor unit included in a bioreactor cluster associated with the target bioreactor unit, except a waste valve for draining the waste from the target bioreactor unit, turning on a corresponding pump for a tank or reservoir storing the waste, monitoring an amount of the waste draining from the target bioreactor unit based on information collected from a flow sensor associated with the corresponding pump, turning off the corresponding pump for the tank or reservoir when a target volume of material is dispensed.

In some embodiments, each bioreactor cluster further includes a top manifold and a plurality of bottom manifolds corresponding to a plurality of rows of bioreactor units included in the cluster. The top manifold further includes a plurality of fluid channels for dispensing the one or more materials into each well, and each bottom manifold further includes one or more fluid channels for draining the waste from each well.

In some embodiments, the plurality of fluid channels for dispensing the one or more materials and the one or more fluid channels for draining the waste are respectively in fluidic communication with the plurality of tanks or reservoirs.

In some embodiments, each of the waste valves and dispensing valves is a binary valve.

In some embodiments, the one or more materials include growth media, oxygen, or auxiliary solution.

### 6. TERMINOLOGY

The phraseology and terminology used herein are for the purpose of description and should not be regarded as limiting.

The phrases “optimizing,” “optimization,” or similar terms can refer to any degree of “improving” or “improvement” to any aspect of an organism, population, or system, including (but not limited to) improving that aspect of the organism, population, or system such that it reaches or approaches an absolute optimum (e.g., absolute maximum or minimum) value.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

What is claimed is:

1. A bioreactor system, comprising:
one or more bioreactor groups, each group having a plurality of bioreactor clusters and a gantry shared by the plurality of bioreactor clusters; wherein
each bioreactor cluster comprises one or more manifolds and a plurality of bioreactor units, each bioreactor unit having a well, a lid covering the well, a waste valve configured to control draining of waste out of the well, and one or more dispensing valves configured to control dispensing of one or more materials into the well, and
the gantry comprises one or more sensors and a movable gantry head with one or more sensor probes configured to selectively sense properties of contents of respective bioreactor units included in the plurality of bioreactor units.

2. The bioreactor system according to claim 1, wherein each bioreactor unit has a respective stirring mechanism, temperature control subsystem, temperature sensor, liquid level sensor, and optical density sensor.

3. The bioreactor system according to claim 1, wherein the plurality of bioreactor units included in each of the bioreactor clusters are organized in a number of rows, each row having a same number of bioreactor units.

4. The bioreactor system according to claim 3, further comprising:
a master controller board configured to control the bioreactor system;
a plurality of cluster controller boards configured to control, respectively, the plurality of bioreactor clusters; and
a plurality of bioreactor boards configured to control, respectively, the rows of bioreactor units.

5. The bioreactor system according to claim 4, wherein the master controller board communicates with the cluster controller board associated with the plurality of bioreactor clusters, and each cluster controller board associated with each bioreactor cluster in the plurality of bioreactor clusters communicates with the bioreactor boards associated with the rows of bioreactor units in the respective bioreactor cluster.

6. The bioreactor system according to claim 3, wherein the one or more manifolds included in each bioreactor cluster comprise a respective top manifold and a respective plurality of bottom manifolds in each bioreactor cluster.

7. The bioreactor system according to claim 6, wherein in each bioreactor cluster, each of the plurality of bottom manifolds corresponds to a row of bioreactor units in the respective bioreactor cluster.

8. The bioreactor system according to claim 6, wherein in each bioreactor cluster, the one or more dispensing valves of the bioreactor units in the respective bioreactor cluster are disposed within the top manifold.

9. The bioreactor system according to claim 6, wherein in each bioreactor cluster, the waste valves of the bioreactor units in each row of the respective bioreactor cluster are disposed within a corresponding bottom manifold.

10. The bioreactor system according to claim 6, wherein in each bioreactor cluster, the top manifold further comprises one or more fluid channels configured to provide the one or more materials to the bioreactor units in the respective bioreactor cluster.

11. The bioreactor system according to claim 10, wherein in each bioreactor cluster, the one or more fluid channels comprise a fluid channel configured to provide growth media to the bioreactor units in the respective bioreactor cluster, a fluid channel configured to provide an auxiliary solution to adjust pH of growth media in the wells of the bioreactor units in the respective bioreactor cluster, and/or a fluid channel configured to provide oxygen to the bioreactor units in the respective bioreactor cluster.

12. The bioreactor system according to claim 9, wherein in each bioreactor cluster, each bottom manifold further comprises a fluid channel configured to remove the waste drained from the bioreactor units in the respective row of the bioreactor cluster corresponding to the respective bottom manifold.

13. The bioreactor system according to claim 1, wherein the one or more sensors included in the gantry comprise a pH sensor, a dissolved oxygen sensor, and/or a spectrometer.

14. The bioreactor system according to claim 1, wherein the lid for each bioreactor unit comprises one or more probe channels for inserting one or more of the pH sensor, the dissolved oxygen sensor, or the spectrometer sensor into the lid from the gantry.

15. The bioreactor system according to claim 14, wherein the lid for each bioreactor unit further comprises one or more stickers configured to facilitate detection of one or more of a pH value or a dissolved oxygen concentration inside the well of the respective bioreactor unit.

16. The bioreactor system according to claim 1, wherein each gantry head is operable to move to a position corresponding to each bioreactor unit included in the respective group of bioreactor clusters corresponding to the gantry head.

17. The bioreactor system according to claim 1, wherein the one or more bioreactor groups comprise a plurality of bioreactor groups stacked vertically to form a bioreactor station.

18. The bioreactor system according to claim 17, comprising 10 bioreactor stations, wherein each bioreactor station comprises four bioreactor groups, each bioreactor group comprises four bioreactor clusters, and each bioreactor cluster comprises 64 bioreactor units.

19. The bioreactor system according to claim 18, wherein the 64 bioreactor units in each bioreactor cluster are organized in an 8×8 array.

20. A method for forming a bioreactor system, comprising:
forming one or more bioreactor groups, each group having a plurality of bioreactor clusters and a gantry shared by the plurality of bioreactor clusters,
wherein each bioreactor cluster comprises one or more manifolds and a plurality of bioreactor units, each bioreactor unit having a well, a lid covering the well, a waste valve configured to control draining of waste out of the well, and one or more dispensing valves configured to control dispensing of one or more materials into the well, and
wherein the gantry comprises one or more sensors and a movable gantry head with one or more sensor probes configured to selectively sense properties of contents of respective bioreactor units included in the plurality of bioreactor units.

21. A method comprising:
in a bioreactor system having one or more bioreactor groups, each group having a plurality of bioreactor clusters and a gantry shared by the plurality of bioreactor clusters, each bioreactor cluster comprising a plurality of bioreactor units, each bioreactor unit having a well, a lid covering the well and having one or more channel windows, and a first set of sensors disposed on one side of the bioreactor unit, and the gantry comprising a second set of sensors and a movable gantry head with one or more sensor probes configured to selectively sense properties of contents of respective bioreactor units included in the plurality of bioreactor units, the method comprising:
performing a first measurement of contents inside a target bioreactor unit using one of the first set of sensors disposed adjacent to the target bioreactor unit; and/or
performing a second measurement of the contents inside the target bioreactor unit using one of the second set of sensors included in the gantry.

22. The method according to claim 21, wherein performing the second measurement of the contents inside the target bioreactor unit using the one of the second set of sensors included in the gantry comprises:
moving the gantry head to a position over the target bioreactor unit;
moving the gantry head towards a lid of the target bioreactor unit, to allow a sensor probe associated with the one of the second set of sensors to insert into a corresponding channel window disposed within the lid of the target bioreactor unit; and
activating the one of the second set of sensors, to conduct the second measurement of the contents inside the target bioreactor unit.

23. The method according to claim 22, wherein the bioreactor system further comprises one X-axis guild rail and two Y-axis linear guide rails associated with the gantry, and wherein moving the gantry head to the position over the target bioreactor unit further comprises:
moving the gantry along the two Y-axis linear guide rails; and/or
moving the gantry along the X-axis guide rail towards the target bioreactor unit.

24. The method according to claim 22, wherein the bioreactor system further comprises a cluster controller board associated with the gantry and a motor to drive the gantry head to move, and wherein moving the gantry head to the position over the target bioreactor unit further comprises:
providing motor drive signals to the gantry head by the cluster controller board, to drive the gantry head to move to the position over the bioreactor unit, wherein the motor drive signals comprise X, Y, and Z position information regarding where the gantry head will move to.

25. The method according to claim 22, wherein the first measurement and second measurement are conducted within a 60 second time period.

26. The method according to claim 21, wherein the first set of sensors comprise a temperature sensor and an optical density sensor, and the second set of sensors comprise a dissolved oxygen sensor, a pH sensor, and a spectrometer sensor.
